SEQUENCE LISTING (22 sequences)

SEQ ID NO: 1 - DNA, 21 bp, Homo sapiens
aatatgtgaagcagacatgga

SEQ ID NO: 2 - DNA, 21 bp, Homo sapiens
aatcaaatcatactccttccc

SEQ ID NO: 3 - DNA, 21 bp, Homo sapiens
aagagagtactgtgtcattca

SEQ ID NO: 4 - DNA, 21 bp, Homo sapiens
aaggaaaacctcatcagggaa

SEQ ID NO: 5 - DNA, 21 bp, Homo sapiens
aatcatactccttcccaaagc

SEQ ID NO: 6 - DNA, 21 bp, Homo sapiens
aattttggaggccgtccacaa

SEQ ID NO: 7 - DNA, 21 bp, Homo sapiens
aagactgggccctttgaggat

SEQ ID NO: 8 - DNA, 21 bp, Homo sapiens
aagcagacatggaacctgcag

SEQ ID NO: 9 - DNA, 21 bp, Homo sapiens
aacttccaggttcccatcctg

SEQ ID NO: 10 - DNA, 21 bp, Homo sapiens
aagcctggccacagaaagtcc

SEQ ID NO: 11 - DNA, 21 bp, Homo sapiens
aagccctacccatggacacag

SEQ ID NO: 12 - DNA, 21 bp, Homo sapiens
aacctgcagggtcaggctctg

SEQ ID NO: 13 - DNA, 21 bp, Homo sapiens
aaggggtgcagcccaagactg

SEQ ID NO: 14 - DNA, 21 bp, Homo sapiens
aacttgcaccctgggctgcag

SEQ ID NO: 15 - DNA, 21 bp, Homo sapiens
aagtcctcccctgcccaaggg

SEQ ID NO: 16 - DNA, 21 bp, Homo sapiens
aaggcccccagagagaagccc

SEQ ID NO: 17 - DNA, 21 bp, Homo sapiens
aatctcaagaatcaaatcata

SEQ ID NO: 18 - DNA, 21 bp, Homo sapiens
aatgttaattttggaggccgt

SEQ ID NO: 19 - DNA, 21 bp, Homo sapiens
aatccgtatgagccagaactt

SEQ ID NO: 20 - DNA, 21 bp, Homo sapiens
aatcggcacacagggaaatgt

SEQ ID NO: 21 - DNA, 21 bp, Homo sapiens
aaccggcaagagagtactgtg

SEQ ID NO: 22 - DNA, 21 bp, Homo sapiens
aaggaggtttacctggaccga

(19) United States
(12) Patent Application Publication
(10) Pub. No.: US 2020/0345737 A1
(43) Pub. Date: Nov. 5, 2020

(54) SYK KINASE INHIBITORS AS TREATMENT FOR MALARIA

(71) Applicant: PURDUE RESEARCH FOUNDATION, WEST LAFAYETTE, IN (US)

(72) Inventors: PHILIP S. LOW, WEST LAFAYETTE, IN (US); FRANCESCO MICHELANGELO TURRINI, TORRINO (IT); KRISTINA ROSE KESELY, LAFAYETTE, IN (US); ANTONELLA PANTALEO, SASSARI (IT)

(21) Appl. No.: 16/793,895

(22) Filed: Feb. 18, 2020

Related U.S. Application Data
(63) Continuation of application No. 14/132,533, filed on Dec. 18, 2013.
(60) Provisional application No. 61/738,888, filed on Dec. 18, 2012.

(51) Int. Cl. A61K 31/519; A61K 31/505; A61K 31/506; A61K 31/366; A61K 31/05; A61K 31/49; A61K 31/675; A61K 31/52; A61K 31/404; A61K 31/4706; A61K 45/06

(52) U.S. Cl. CPC A61K 31/519; A61K 31/505; A61K 31/506; A61K 31/366; A61K 31/05; A61K 45/06; A61K 31/675; A61K 31/52; A61K 31/404; A61K 31/4706; A61K 31/49

(57) ABSTRACT

The disclosure relates to methods, compositions, and kits for treatment of parasite-mediated disease. In one embodiment, the disclosure relates to compounds, compositions, methods and kits for the treatment of malaria. In still another embodiment, the disclosure relates to a method for treating malaria comprising the use of a Syk kinase inhibitor.

### CROSS REFERENCE TO RELATED APPLICATIONS

This application is continuation of U.S. application Ser. No. 14/132,533, filed Dec. 18, 2013, which is a non-provisional patent application of and claims priority to Provisional Patent Application No. 61/738,888 filed Dec. 18, 2012, which is incorporated herein by reference in their entirety.

### STATEMENT OF GOVERNMENT RIGHTS

This invention was made with government support under GM024417 awarded by The National Institutes of Health. The government has certain rights in the invention.

### FIELD

Embodiments of the disclosure relate to malaria. More specifically, embodiments of the disclosure are related to methods, compositions and kits for treatment of malaria. In one embodiment, the disclosure relates to methods of treating malaria using a Syk kinase inhibitor.

### BACKGROUND

Despite intense world-wide effort, malaria is still a major cause of mortality and morbidity, especially in third world countries. According to the World Health Organization's (WHO) “WHO Malaria World Report 2011,” there were approximately 216 million episodes of the disease in 2010, which resulted in 655,000 deaths. Approximately 81% of cases were in the African Region, about 91% being due to P. falciparum.

Malaria infections are particularly lethal to young children with 86% of the malaria's victims being under the age of 5. While the annual incidences and mortalities have dropped by 17% and 26% respectively since 2000, this is far short of the targeted goal of a 50% reduction as proposed in the initial Global Malaria Action Plan of the Roll Back Malaria Partnership. Furthermore, drug resistant strains are emerging in several parts of the world. In Cambodia, Myanmar, Thailand and Vietnam, malarial strains resistant to the primary treatment therapy, artemisinin, are stimulating efforts to contain the spread of these resistant forms.

One method recommended by WHO for dealing with these drug resistant strains is to replace artemisinin-based monotherapies with combination therapies that include artemisinin (ACTs). Another method would be to develop a drug that could not be evaded by a mutation in the parasite; i.e. identify a drug that blocks a critical step in parasite maturation performed only by the host erythrocyte. In either strategy, new drug treatment strategies are required.

As the malaria-inducing vector, the Plasmodium's life cycle provides several potential targets for therapeutic intervention [JCI 2008, 118:4, 1266-1276]. Infection is initiated when a mosquito transfers Plasmodium sporozoites into a mammalian host while collecting its own blood meal. A portion of these sporozoites migrate from the dermis into the bloodstream where they travel to the liver and invade hepatocytes, thereby initiating the developmental phase of their life cycle [Curr Opin in Micro 2008, 11:352-359].

After the sporozoites enter the hepatocytes, they create a nascent parasitophorous vesicle in which the parasite enters a phase of cell division resulting in the formation of merozoites. Eventually these liver merozoites are released back in to bloodstream in membrane encapsulated structures called merosomes, where they travel through the body until eventually bursting and releasing into the blood. These free merozoites then randomly adhere to erythrocytes, orient themselves with the apical surface attached to the red blood cell membrane, invade the host RBC while shedding their surface coat outside the cell, and initiate their asexual phase of replication. After the merozoite enters the red blood cell (RBC), a large digestive vacuole forms where the parasite digests hemoglobin to provide the amino acids necessary for protein biosynthesis (Ring Stage). The parasite then begins to express its own proteins as it progresses through the trophozoite (expansion) stage, and after DNA replication, into schizont phase where approximately 16 fully formed merozoites are generated within the RBC.

During this process, the RBC membrane is gradually weakened in preparation for egress of the parasite from the RBC and for infecting additional erythrocytes. Culmination of this egress phase occurs when the protective parasitophorous vesicle bursts within the RBC, triggering swelling of the host cell, degradation of the cytoskeleton, and eventually rupture of the host RBC membrane, releasing the free merozoites into circulation [Cell 2006, 124:755-766]. These merozoites are then able to infect erythrocyte hosts and initiate a new cycle. While most events in this life cycle are controlled by parasite-encoded proteins, a few critical events depend entirely on RBC components, opening a window of opportunity for designing a mutation proof therapy for malaria.

During the course of the parasite life cycle within the mammalian host, there are dramatic changes in protein phosphorylation of parasite and erythrocyte proteins. Many of these phosphorylation events are life cycle stage specific and are therefore likely to be critical in the parasite maturation process [Nature Comm 2011, 2:565-578; Microbes and Infection, 2012, epub (Lasonder E); J. Proteomics, 2009, 73: 445-455]. Indeed, non-specific kinase inhibitors have shown efficacy in blocking parasite maturation in vitro.

However, due to their ubiquitous nature, these inhibitors are too toxic for clinical use. Further, inhibitors that block parasite protein kinase activity would seem susceptible to the mutagenic events that are associated with the development of disease resistance. Interestingly, while parasite infection is associated with changes in both serine/threonine and tyrosine phosphorylation, the parasite genome does not express any tyrosine kinases [Biochim Biophy Acta 2005, 1754:132-150; Trends in Parasitology 2008, 24: 570-577]. Therefore, the inventors speculate that the tyrosine phosphorylation that occurs during the latter stages of the parasite life cycle within the RBC must be induced by erythrocyte tyrosine kinases. Specific inhibition of erythrocyte tyrosine kinases that are critical in the parasite life cycle could be a valuable therapeutic target for blocking parasite propagation while evading resistant mechanisms associated with mutagenic events within the parasite genome.

Several studies have identified proteins that are phosphorylated upon malarial infection [BBA, 1990, 1053:118-124; Mol Biochem Parasit, 1989, 34:229-236; Exp Parasitol 1998, 89:40-49; Blood 2005, 106: 4359-4366; Mol Biochem Parasitol 1991, 46:113-122; Malaria J 2009, 8:105-122]. Band 3 represents the earliest and most prominent tyrosine phosphorylation event during parasite development, beginning at low levels during early ring stage parasitemia and increasing continuously until parasite egress [Proteomics 2010, 10:3469-3479]. Interestingly, deletion of the amino terminal 11 amino acids of band 3 results in a decrease in band 3 tyrosine phosphorylation and a significant reduction in the ability of P. falciparum to infect red blood cells [Blood 2005, 106:4359-4366]. Similarly, red blood cells isolated from individuals with Southeast Asian ovalocytosis contain a mutated form of band 3 with a 9 amino acid deletion and appear to be protected against development of the cerebral form of the disease [Nature 1995, 378:564-565; Am J Trop Med Hyg 1999, 60: 1056-1060; Mol Biochem Parasitol 2006, 149:121-127].

Band 3 tyrosine phosphorylation significantly impacts erythrocyte function in a number of fashions. Band 3 organizes a complex of glycolytic enzymes on the membrane and thereby controls the flux of glucose between the pentose phosphate pathway (PPP) and glycolysis. Syk phosphorylation of band 3 leads to displacement of these glycolytic enzymes from an inhibitory site on band 3, resulting in activation of glycolysis. This activation shifts the consumption of glucose from PPP to glycolysis, resulting in a decline in RBC reducing power and a concomitant increase in RBC ATP. Phosphorylation of band 3 has also been shown to inhibit band 3 mediated anion transport.

However, since this inhibition is relatively mild, it seems unlikely that this phosphorylation activity has much impact on parasitemia. Band 3 is prominently engaged in anchoring of the membrane cytoskeleton to the lipid bilayer. This band 3 function is linked specifically to its association with adducin at the junctional complex and ankyrin at the ankyrin complex. Rupture of either of these two bridges yields an erythrocyte that spontaneously loses membrane surface through vesiculation/blebbing. Recent studies from our lab demonstrate that Syk-mediated tyrosine phosphorylation of band 3 leads to complete inhibition of ankyrin binding and the consequent dissociation of band 3 from the cytoskeleton. When induced in freshly isolated erythrocytes in suspension, tyrosine phosphorylation of band 3 surprisingly results in major changes in RBC morphology without loss of membrane surface area. However, when the same RBCs are even slightly mechanically agitated (e.g. rocking the suspension), they immediately bleb membrane surface and vesiculate. Indeed, membrane vesiculation in vivo constitutes a common characteristic of erythrocyte pathologies (sickle cell disease, G6PDH deficiency, β-thalassemia) that are characterized by elevated band 3 tyrosine phosphorylation. In these diseases, the released microparticles, as they are termed in the literature, are thought to promote thrombosis and its associated morbidities.

The fact that certain mutations involving erythrocyte enzymes (G6PD, PK deficiency), hemoglobin (thalassemias, sickle cell disease, HbC, etc) or membrane proteins (Southeast Asian ovalocytosis, spherocytosis, etc) can protect against malaria constitutes very strong evidence that a dysfunction in host cell functions can establish stable protection against malaria. Those mutations are effective in the heterozygous state and are generally asymptomatic. Drugs that target erythrocyte functions may possess the advantage to elude the resistance mechanisms derived from parasite mutational changes. In other words, drugs that target parasite structures can be rendered ineffective by mutations in the parasite that bypass the effect of the drug. Conversely, drugs that primarily target erythrocyte components, which are critical for maturation, survival or egress of the parasite, should not be vulnerable to drug resistance mechanisms.

Therefore, chemical compounds, drugs, compositions and methods that target erythrocyte components are needed in order to develop therapies that are not vulnerable to drug resistance.

### BRIEF SUMMARY

In one embodiment, the disclosure relates to method of treating malaria comprising administering an effective amount of a Syk kinase inhibitor to a subject in need thereof. In some embodiments, the Syk kinase inhibitor is selective for Syk kinase, thereby specifically targeting Syk kinase activity.

In one aspect, the disclosure provides methods of inhibiting SYK signaling in vivo or in vitro, comprising administering an effective amount of a Syk kinase inhibitor.

In one aspect, the disclosure provides methods for treating malaria comprising administering to a subject in need of such treatment a therapeutically effective amount of a Syk kinase inhibitor or pharmaceutically acceptable salts, pharmaceutical compositions or medicaments thereof.

In one embodiment, the disclosure relates to a method for treating malaria comprising: (a) identifying a patient in need of treatment from malaria; and (b) administering to said patient a therapeutically effective amount of a Syk kinase inhibitor to treat malaria.

In still another embodiment, the disclosure relates to a method for reducing the incidence of malaria comprising: (a) identifying a subject who may be a carrier of malaria; and (b) administering a therapeutically effective amount of a Syk kinase inhibitor to said subject.

In yet another embodiment, the disclosure relates to a method for treating drug resistant malaria comprising (a) identifying a patient with drug resistant malaria; and (b) administering to said patient a therapeutically effective amount of a Syk kinase inhibitor to treat malaria.

In another embodiment, the disclosure relates to a method for treating a parasite-mediated disease in a patient in need thereof comprising: administering to said patient a therapeutically effective amount of a Syk kinase inhibitor.

In still another embodiment, the disclosure relates to a method for inhibiting rupture of a red blood cell comprising contacting a red blood cell with an Syk kinase inhibitor, or a pharmaceutically acceptable salt thereof.

In still another embodiment, the disclosure relates to a method for treating malaria comprising administering a tyrosine kinase inhibitor that targets an erythrocyte or RBC component.

In yet another embodiment, the Syk kinase inhibitor is selected from the group consisting of Syk kinase inhibitor II, Syk kinase inhibitor IV, imatinib mesylate and combinations thereof.

In another embodiment, the Syk kinase inhibitor is selected from the group consisting of a purine-2-benzamine derivative, a pyrimidine-5-carboxarnide derivative, a 1,6-naphthyridine derivative, BAY 61-3606, piceatannol, 3,4-dimethyl-10-(3-aminopropyl)-9-acridone oxalate), R406, R788, and combinations thereof.

In another embodiment, the Syk kinase inhibitor is administered or used with one or more antimalarial drugs. In another embodiment, the antimalarial drug is selected from the group consisting of: artimisinin, chloroquine, quninine, and indolone N-oxides (INODS) of various structures.

In another aspect, the disclosure provides a medicament for treating a Syk-mediated disease, disorder or condition comprising a therapeutically effective amount of a Syk kinase inhibitor. In one embodiment, the Syk-mediated disease is malaria.

In another aspect, the disclosure provides the use of a Syk kinase inhibitor in the manufacture of a medicament for treating a SYK-mediated disease, disorder or condition.

In certain embodiment, the kinase inhibitor effectively inhibits activity of one or more kinases selected from SYK, PYK2, FAK, ZAP70, PIM1, FLT3, RET, JAK2, JAK3, LRRK2, LRRK2(G2019S), ALK, AURKA, AXL, BMPR2, CSFIR, JNK1, JNK2, JNK3, KIT, KIT(D816V), LKB1, MLK1, PAK4, PDGFRB, PLK4, RSK2, SNARK, SRPK3, TAK1, and TYK2.

In another aspect, the disclosure provides methods for treating a protein kinase-mediated rupture of a cell comprising administering to a subject in need of such treatment a therapeutically effective amount of a kinase inhibitor or a pharmaceutically acceptable salt, a pharmaceutical composition or a medicament thereof. The protein kinase includes, but is not limited to, SYK, PYK2, FAK, ZAP70, PIM1, FLT3, RET, JAK2, JAK3, LRRK2, LRRK2(G2019S), ALK, AURKA, AXL, BMPR2, CSF1R, JNK1, JNK2, JNK3, KIT, KIT(D816V), LKB1, MLK1, PAK4, PDGFRB, PLK4, RSK2, SNARK, SRPK3, TAK1, and TYK2.

In another embodiment, the disclosure relates to a method of inhibiting Syk kinase expression using small interfering RNA (siRNA) and to therapeutic strategies based on such a method.

In yet another embodiment, the disclosure relates to methods for treating malaria comprising administering to a subject in need of such treatment a therapeutically effective amount of a siRNA directed to Syk or pharmaceutically acceptable salts, pharmaceutical compositions or medicaments thereof.

The methods and compositions disclosed herein offer distinct advantages over other therapies including: (i) the mechanism of Plasmodium suppression is totally distinct from any previous therapy examined, suggesting prior drug resistance mechanisms will not be functional; (ii) the therapy involves inhibition of an RBC tyrosine kinase that has no counterpart in the parasite genome, rendering escape mutations that might lead to disease resistance very difficult, and (iii) one of the Syk kinase inhibitors shown to be effective is currently in FDA-approved clinical trials for daily use for treatment of rheumatoid arthritis patients, suggesting the drug is readily tolerated.

An advantage of the methods and compositions disclosed herein is that the targeted enzyme belongs to the red blood cell, and thus, the parasite cannot mutate to avoid the therapy.

An advantage of the methods and compositions disclosed herein is that the target of the therapeutic intervention has no counterpart in the parasite genome.

An advantage of the methods and compositions disclosed herein is that the parasite does not contain a tyrosine kinase.

### BRIEF DESCRIPTION OF THE DRAWINGS

FIG. 1 is a photograph demonstrating tyrosine phosphorylation of band 3 during P. falciparum growth in human erythrocytes. Western blot of the 100 kDa region stained with anti-band 3 and anti-phosphotyrosine antibodies in the presence or in the absence of Syk kinase inhibitor s PP1/PP2. Samples analyzed were RBC membrane proteins from control (C), ring stage (R) and trophozoite stage (T) of P. falciparum infected RBCs. Proteins were separated by on a 10% SDS-PAGE gel.

FIG. 2 is a line graph depicting the effect of Syk kinase inhibitor II on Parasitemia.

FIG. 3 is a line graph depicting the efficiency of Syk kinase inhibitor (added at different stages of parasite development) on parasite re-infection. Syk kinase inhibitor concentration 1 μM. The inhibitors have been added at different times following the start of the parasite cultures (12, 24, 36, 40, 48 hours). Growth inhibition is calculated as % of the re-invasion rate measured in untreated cultures. The efficiency of the inhibitors added at different times is expressed as a % of the time that determined the maximal inhibition of the re-infection rates. Data are mean of 2 experiments with SD.

FIGS. 4A-4B are line graphs depicting the result of Syk kinase inhibitors on vesiculation and loss of band 3 protein from the erythrocyte membrane. FIG. 4A depicts the results of the control parasite cultures. FIG. 4B depicts the results of the parasite cultures treated with a Syk kinase inhibitor.

FIG. 5 is a schematic depicting the interaction between band 3 and ankyrin and the effects of phosphorylation.

FIG. 6 is a bar graph showing the effect of Syk kinase inhibitor IV on parasite re-invasion.

FIG. 7 is a photograph showing that imatinib mesylate (Gleevec®) is a Syk kinase inhibitor. Effector induced tyrosine phosphorylation of erythrocyte membrane protein band 3 is inhibited when red blood cells are treated with imatinib mesylate (Gleevec®).

FIG. 8 is a line graph demonstrating that imatinib mesylate (Gleevec®) inhibits Plasmodium falciparum growth in vitro at clinically relevant concentrations.

FIGS. 9A-9E are microscopy photographs of untreated red blood cells and red blood cells treated with Gleevec®.

FIGS. 10A-10D are microscopy photographs of untreated red blood cells and red blood cells treated with Gleevec®.

FIGS. 11A-11C are microscopy photographs of red blood cells treated with Gleevec®.

FIG. 12 is a bar graph showing the percent reduction in parasitemia in blood from Vietnamese patients at different concentrations of Syk kinase inhibitor II.

FIG. 13 is a bar graph showing the percent reduction in parasitemia in blood from Vietnamese patients at different concentrations of imatinib mesylate (Gleevec®) ex vivo.

### DETAILED DESCRIPTION

### Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide one of skill with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range.

The headings provided herein are not limitations of the various aspects or embodiments of this disclosure, which can be had by reference to the specification as a whole. Accordingly, the terms defined immediately below are more fully defined by reference to the specification as a whole.

It is noted that, as used in the specification and the appended claims, the singular forms “a,” “an” and “the” include plural referents unless the context clearly dictates otherwise. Further, definitions of standard chemistry terms may be found in reference works, including Carey and Sundberg “ADVANCED ORGANIC CHEMISTRY 4.sup.TH ED” Vols. A (2000) and B (2001), Plenum Press, New York. Also, unless otherwise indicated, conventional methods of mass spectroscopy, NMR, HPLC, protein chemistry, biochemistry, recombinant DNA techniques and pharmacology, within the skill of the art are employed.

The numerical ranges in this disclosure are approximate, and thus may include values outside of the range unless otherwise indicated. Numerical ranges include all values from and including the lower and the upper values, in increments of one unit, provided that there is a separation of at least two units between any lower value and any higher value. As an example, if a compositional, physical or other property, such as, for example, molecular weight, viscosity, etc., is from 100 to 1,000, it is intended that all individual values, such as 100, 101, 102, etc., and sub ranges, such as 100 to 144, 155 to 170, 197 to 200, etc., are expressly enumerated. For ranges containing values which are less than one or containing fractional numbers greater than one (e.g., 1.1, 1.5, etc.), one unit is considered to be 0.0001, 0.001, 0.01 or 0.1, as appropriate. For ranges containing single digit numbers less than ten (e.g., 1 to 5), one unit is typically considered to be 0.1. These are only examples of what is specifically intended, and all possible combinations of numerical values between the lowest value and the highest value enumerated, are to be considered to be expressly stated in this disclosure. Numerical ranges are provided within this disclosure for, among other things, relative amounts of components in a mixture, and various temperature and other parameter ranges recited in the methods.

As used herein, “Cₘ to Cₙ” in which “m” and “n” are integers refers to the number of carbon atoms in an alkyl, alkenyl or alkynyl group or the number of carbon atoms in the ring of a cycloalkyl, cycloalkenyl, cycloalkynyl, aryl, heteroaryl or heteroalicyclyl group. That is, the alkyl, alkenyl, alkynyl, ring of the cycloalkyl, ring of the cycloalkenyl, ring of the cycloalkynyl, ring of the aryl, ring of the heteroaryl or ring of the heteroalicyclyl can contain from “m” to “n”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “m” and “n” are designated with regard to an alkyl, alkenyl, alkynyl, cycloalkyl cycloalkenyl, cycloalkynyl, aryl, heteroaryl or heteroalicyclyl group, the broadest range described in these definitions is to be assumed.

As used herein, the term “administering” refers to oral administration, administration as a suppository, topical contact, intravenous, intraperitoneal, intramuscular, intralesional, intranasal or subcutaneous administration, or the implantation of a slow-release device e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intraarteriole, intradermal, subcutaneous, intraperitoneal, intraventricular, and intracranial. Other modes of delivery include, but are not limited to, the use of liposomal formulations, intravenous infusion, transdermal patches, etc.

As used herein, “alkyl” refers to a straight or branched hydrocarbon chain fully saturated (no double or triple bonds) hydrocarbon group. The alkyl group may have 1 to 50 carbon atoms (whenever it appears herein, a numerical range such as “1 to 50” refers to each integer in the given range; e.g., “1 to 50 carbon atoms” means that the alkyl group may consist of 1 carbon atom, 2 carbon atoms, 3 carbon atoms, etc., up to and including 50 carbon atoms, although the present definition also covers the occurrence of the term “alkyl” where no numerical range is designated). The alkyl group may also be a medium size alkyl having 1 to 30 carbon atoms. The alkyl group could also be a lower alkyl having 1 to 5 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl and the like.

The alkyl group may be substituted or unsubstituted. When substituted, the substituent group(s) is(are) one or more group(s) individually and independently selected from alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl (mono-, di- and tri-substituted haloalkyl), haloalkoxy (mono-, di- and tri-substituted haloalkoxy), trihalomethanesulfonyl, trihalomethanesulfonamido, and amino, including mono- and di-substituted amino groups, and the protected derivatives thereof.

As used herein, “alkenyl” refers to an alkyl group that contains in the straight or branched hydrocarbon chain one or more double bonds. An alkenyl group may be unsubstituted or substituted. When substituted, the substituent(s) may be selected from the same groups disclosed above with regard to alkyl group substitution unless otherwise indicated.

As used herein, the term “analogs” refers to compounds that are substantially the same as another compound but which may have been modified by, for example, adding side groups, oxidation or reduction of the parent structure. Analogs of the Syk kinase inhibitors disclosed herein can be readily prepared using commonly known standard reactions. These standard reactions include, but are not limited to, hydrogenation, alkylation, acetylation, and acidification reactions. Chemical modifications can be accomplished by those skilled in the art by protecting all functional groups present in the molecule and deprotecting them after carrying out the desired reactions using standard procedures known in the scientific literature (Greene, T. W. and Wuts, P. G. M. “Protective Groups in Organic Synthesis” John Wiley & Sons, Inc. New York. 3rd Ed. pg. 819, 1999; Honda, T. et al. Bioorg. Med. Chem. Lett., 1997, 7:1623-1628; Honda, T. et al. Bioorg. Med. Chem. Lett., 1998, 8:2711-2714; Konoike, T. et al. J Org. Chem., 1997, 62:960-966; Honda, T. et al. J. Med. Chem., 2000, 43:4233-4246; each of which are hereby incorporated herein by reference in their entirety). Analogs exhibiting the desired biological activity (such as inhibition of an HDAC, potential to differentiate cells, etc.) can be identified or confirmed using cellular assays or other in vitro or in vivo assays.

As used herein, “alkynyl” refers to an alkyl group that contains in the straight or branched hydrocarbon chain one or more triple bonds. An alkynyl group may be unsubstituted or substituted. When substituted, the substituent(s) may be selected from the same groups disclosed above with regard to alkyl group substitution unless otherwise indicated.

As used herein, “aryl” refers to a carbocyclic (all carbon) monocyclic or multicyclic aromatic ring system that has a fully delocalized pi-electron system. Examples of aryl groups include, but are not limited to, benzene, naphthalene and azulene. The ring of the aryl group may have 5 to 50 carbon atoms. The aryl group may be substituted or unsubstituted. When substituted, hydrogen atoms are replaced by substituent group(s) that is(are) one or more group(s) independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxy, alkoxy, aryloxy, acyl, ester, mercapto, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl (mono-, di- and tri-substituted haloalkyl), haloalkoxy (mono-, di- and tri-substituted haloalkoxy), trihalomethanesulfonyl, trihalomethanesulfonamido, and amino, including mono- and di-substituted amino groups, and the protected derivatives thereof, unless the substituent groups are otherwise indicated.

As used herein, “cell” or “cells,” unless specifically limited to the contrary, includes any somatic cell, embryonic stein (ES) cell, adult stem cell, an organ specific stem cell, nuclear transfer (NT) units, and stem-like cells. The cell or cells can be obtained from any organ or tissue. The cell or cells can be human or other animal. For example, a cell can be mouse, guinea pig, rat, cattle, horses, pigs, sheep, goats, etc. A cell also can be from non-human primates.

As used herein, “cycloalkenyl” refers to a cycloalkyl group that contains one or more double bonds in the ring although, if there is more than one, the double bonds cannot form a fully delocalized pi-electron system in the ring (otherwise the group would be “aryl,” as defined herein). When composed of two or more rings, the rings may be connected together in a fused, bridged or spiro-connected fashion. A cycloalkenyl group may be unsubstituted or substituted. When substituted, the substituent(s) may be an alkyl or selected from the substituents disclosed above with respect to alkyl group substitution unless otherwise indicated.

As used herein, “cycloalkynyl” refers to a cycloalkyl group that contains one or more triple bonds in the ring. When composed of two or more rings, the rings may be joined together in a fused, bridged or spiro-connected fashion. A cycloalkynyl group may be unsubstituted or substituted. When substituted, the substituent(s) may be an alkyl or selected from the substituents disclosed above with respect to alkyl group substitution unless otherwise indicated.

As used herein, “cycloalkyl” refers to a completely saturated (no double bonds) mono- or multi-cyclic hydrocarbon ring system. When composed of two or more rings, the rings may be joined together in a fused, bridged or spiro-connected fashion. Cycloalkyl groups may range from C3 to Cm, in other embodiments it may range from C₃ to C₈. A cycloalkyl group may be unsubstituted or substituted. Typical cycloalkyl groups include, but are in no way limited to, cyclopropyl, cyclobutyl, cyclopentyl, cyclohexyl, and the like. If substituted, the substituent(s) may be an alkyl or selected from those substituents indicated above with respect to substitution of an alkyl group unless otherwise indicated.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” includes trifluoromethyl, 2,2,2-trifluoroethyl, 4-chlorobutyl, 3-bromopropyl, and the like.

As used herein, “heterocyclyl” and “heteroalicyclyl” refer to a stable 3- to 18 membered ring that consists of carbon atoms and from one to five heteroatoms selected from the group consisting of nitrogen, oxygen and sulfur. The “heterocyclyl” or “heteroalicyclyl” may be monocyclic, bicyclic, tricyclic, or tetracyclic ring system, which may be joined together in a fused, bridged or spiro-connected fashion; and the nitrogen, carbon and sulfur atoms in the “heterocyclyl” or “heteroalicyclyl” may be optionally oxidized; the nitrogen may be optionally quaternized; and the rings may also contain one or more double bonds provided that they do not form a fully delocalized pi-electron system throughout all the rings. Heterocyclyl and heteroalicyclyl groups may be unsubstituted or substituted. When substituted, the substituent(s) may be one or more groups independently selected from the group consisting of alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, haloalkyl (mono-, di- and tri-substituted haloalkyl), haloalkoxy (mono-, di- and tri-substituted haloalkoxy), trihalomethanesulfonyl, trihalomethanesulfonamido, and amino, including mono- and di-substituted amino groups, and the protected derivatives thereof. Examples of such “heteroalicyclic” or “heteroalicyclyl” include but are not limited to, azepinyl, acridinyl, carbazolyl, cinnolinyl, 1,3-dioxin, 1,3-dioxane, 1,4-dioxane, 1,2-dioxolanyl, 1,3-dioxolanyl, 1,4-dioxolanyl, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazolinyl, imidazolidine, isoxazoline, isoxazolidine, oxazoline, oxazolidine, oxazolidinone, thiazoline, thiazolidine, morpholinyl, oxiranyl, piperidinyl N-Oxide, piperidinyl, piperazinyl, pyrrolidinyl, pyrrolidone, pyrrolidione, 4-piperidonyl, pyrazoline, pyrazolidinyl, 2-oxopyrrolidinyl, tetrahydropyran, 4H-pyran, tetrahydrothiopyran, thiamorpholinyl, thiamorpholinyl sulfoxide, thiamorpholinyl sulfone, and their benzo-fused analogs (e.g., benzimidazolidinone, tetrahydroquinoline, 3,4-methylenedioxyphenyl).

As used herein, “heteroalkyl” refers to an alkyl group as described herein in which one or more of the carbons atoms in the backbone of alkyl group has been replaced by a heteroatom such as nitrogen, sulfur and/or oxygen.

As used herein, “heteroalkenyl” refers to an alkenyl group as described herein in which one or more of the carbons atoms in the backbone of alkenyl group has been replaced by a heteroatom, for example, nitrogen, sulfur and/or oxygen.

As used herein, “heteroalkynyl” refers to an alkynyl group as described herein in which one or more of the carbons atoms in the backbone of alkynyl group has been replaced by a heteroatom such as nitrogen, sulfur and/or oxygen.

As used herein, “heteroaryl” refers to a monocyclic or multicyclic aromatic ring system (a ring system with fully delocalized pi-electron system) that contain(s) one or more heteroatoms, that is, an element other than carbon, including but not limited to, nitrogen, oxygen and sulfur. The ring of the heteroaryl group may have 5 to 50 atoms. The heteroaryl group may be substituted or unsubstituted. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline, and triazine. A heteroaryl group may be substituted or unsubstituted. When substituted, hydrogen atoms are replaced by substituent group(s) that is(are) one or more group(s) independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxy, alkoxy, aryloxy, acyl, ester, mercapto, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl (mono-, di- and tri-substituted haloalkyl), haloalkoxy (mono-, di- and tri-substituted haloalkoxy), trihalomethanesulfonyl, trihalomethanesulfonamido, and amino, including mono- and di-substituted amino groups, and the protected derivatives thereof.

In another aspect, to “inhibit” is to destroy, prevent, control, decrease, slow or otherwise interfere with the growth or survival of a pathogen by at least about 1-fold or more, for example, about 1.5-fold to about 100-fold, or any value in between for example by at least about 2.0, 2.5, 3.0, 3.5, 4.0, 4.5, 5.0, 5.5, 6.0, 6.5, 7.0, 7.5, 8.0, 8.5, 9.0, 9.5, 10, 15, 20, 25, 30, 35, 40, 45, 50, 55, 60, 65, 70, 75, 80, 85, 90, 95-fold when compared to the growth or survival of the pathogen in an untreated control.

In one embodiment, inhibition of the growth of pathogen occurs immediately. In another aspect, inhibition of the growth of pathogen occurs one minute after, 30 minutes after, 45 minutes after, one hour after, two hours after, four hours after, six hours after, twelve hours after, eighteen hours after, or one day after a bacterial strain or composition disclosed herein is applied to a plant material.

In one aspect, inhibition of the growth of pathogen lasts for or provides protection for greater than one or more days, two or more days, three or more days, four or more days, five or more days, one week, two weeks, three weeks, or one month after a bacterial strain or composition disclosed herein is applied to the subject material. In another embodiment, inhibition of the growth of pathogen lasts from one to seven days, from seven to 14 days, from 14 to 21 days, or from 21 to 30 days. In another embodiment, inhibition of pathogen growth lasts until a plant material is consumed or discarded.

As used herein, “small molecule” refers to a non-peptidic, non-oligomeric organic compound either synthesized in the laboratory or found in nature. Small molecules, as used herein, can refer to compounds that are “natural product-like,” however, the term “small molecule” is not limited to “natural product-like” compounds. Rather, a small molecule is typically characterized in that it possesses one or more of the following characteristics including having several carbon-carbon bonds, having multiple stereocenters, having multiple functional groups, having at least two different types of functional groups, and having a molecular weight of less than 1500, although this characterization is not intended to be limiting for the purposes of the disclosure.

As used herein, “substituent convertible to hydrogen in vivo” means any group that is convertible to a hydrogen atom by enzymological or chemical means including, but not limited to, hydrolysis and hydrogenolysis. Examples include hydrolyzable groups, such as acyl groups, groups having an oxycarbonyl group, amino acid residues, peptide residues, o-nitrophenylsulfenyl, trimethylsilyl, tetrahydro-pyranyl, diphenylphosphinyl, and the like. Examples of acyl groups include formyl, acetyl, trifluoroacetyl, and the like. Examples of groups having an oxycarbonyl group include ethoxycarbonyl, t-butoxycarbonyl [(CH₃)₃C—OCO—], benzyloxycarbonyl, p-methoxybenzyloxycarbonyl, vinyloxycarbonyl-β-(p-toluenesulfonyl)ethoxycarbonyl, and the like. Examples of suitable amino acid residues include amino acid residues per se and amino acid residues that are protected with a protecting group. Suitable amino acid residues include, but are not limited to, residues of Gly (glycine), Ala (alanine); Arg (arginine), Asn (asparagine), Asp (aspartic acid), Cys (cysteine), Glu (glutamic acid), His (histidine), Ile (isoleucine), Leu (leucine), Lys (lysine), Met (methionine), Phe (phenylalanine), Pro (praline), Ser (serine), Thr (threonine), Trp (tryptophan), Tyr (tyrosine), Val (valine), Nva (norvaline), Hse (homoserine), 4-Hyp (4-hydroxyproline), 5-Hyl (5-hydroxylysine), Orn (ornithine) and p-Ala. Examples of suitable protecting groups include those typically employed in peptide synthesis, including acyl groups (such as formyl and acetyl), arylmethyloxycarbonyl groups (such as benzyloxycarbonyl and p-nitrobenzyloxycarbonyl), t-butoxycarbonyl groups [(CH₃)₃C—OCO—], and the like.

Suitable peptide residues include peptide residues comprising two to five, and optionally two to three, of the aforesaid amino acid residues. Examples of such peptide residues include, but are not limited to, residues of such peptides as Ala-Ala [CH₃CH(NH₂)CO—NHCH(CH₃)CO—], Gly-Phe, Nva-Nva, Ala-Phe, Gly-Gly, Gly-Gly-Gly, Ala-Met, Met-Met, Leu-Met and Ala-Leu. The residues of these amino acids or peptides can be present in stereochemical configurations of the D-form, the L-form or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr and Tyr. Peptide residues having an asymmetric carbon atom include peptide residues having one or more constituent amino acid residues having an asymmetric carbon atom.

Examples of suitable amino acid protecting groups include those typically employed in peptide synthesis, including acyl groups (such as formyl and acetyl), arylmethyloxycarbonyl groups (such as benzyloxycarbonyl and p-nitrobenzyloxycarbonyl), t-butoxycarbonyl groups [(CH₃)₃C—OCO—], and the like. Other examples of substituents “convertible to hydrogen in vivo” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trityl and benzyloxymethyl); arylmethoxycarbonyl groups (such as benzyloxycarbonyl and o-methoxy-benzyloxycarbonyl); and halogenoethoxycarbonyl groups.

“Substituted or unsubstituted” means that a given moiety may consist of only hydrogen substituents through available valencies (unsubstituted) or may further comprise one or more non-hydrogen substituents through available valencies (substituted) that are not otherwise specified by the name of the given moiety. For example, isopropyl is an example of an ethylene moiety that is substituted by —CH₃. In general, a non-hydrogen substituent may be any substituent that may be bound to an atom of the given moiety that is specified to be substituted. Examples of substituents include, but are not limited to, aldehyde, alicyclic, aliphatic, (C₁₋₁₀)alkyl, alkylene, alkylidene, amide, amino, aminoalkyl, aromatic, aryl, bicycloalkyl, bicycloaryl, carbamoyl, carbocyclyl, carboxyl, carbonyl group, cycloalkyl, cycloalkylene, ester, halo, heterobicycloalkyl, heterocycloalkylene, heteroaryl, heterobicycloaryl, heterocycloalkyl, oxo, hydroxy, iminoketone, ketone, nitro, oxaalkyl, and oxoalkyl moieties, each of which may optionally also be substituted or unsubstituted.

In one particular embodiment, examples of substituents include, but are not limited to, hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloakyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂) bicycloaryl.

In addition, the substituent is itself optionally substituted by a further substituent. In one embodiment, examples of the further substituent include, but are not limited to, hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂) bicycloaryl.

As used herein, “mammal” includes, without limitation, humans, domestic animals (e.g., dogs or cats), farm animals (cows, horses, or pigs), monkeys, rabbits, mice, and laboratory animals.

As used herein, “malaria” is a parasitic disease that involves high fevers, shaking chills, flu-like symptoms, and anemia. Malaria includes but is not limited to Quartan malaria, Falciparum malaria, Biduoterian fever, Blackwater fever, Tertian malaria, Plasmodium, uncomplicated malaria and severe malaria.

As used herein, “patient” refers to human and non-human animals, especially mammals. Examples of patients include, but are not limited to, humans, apes, cows, dogs, cats, goats, sheep, pigs and rabbits.

The term “pharmaceutically acceptable carrier or excipient” means a carrier or excipient that is useful in preparing a pharmaceutical composition that is generally safe, non-toxic and neither biologically nor otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

The terms “pharmaceutically effective amount,” “therapeutically effective amount,” or “therapeutically effective dose” refers to the amount of the subject compound that will elicit the biological or medical response of a tissue, system, animal or human that is being sought by the researcher, veterinarian, medical doctor or other clinician. The term “therapeutically effective amount” includes that amount of a compound that, when administered, is sufficient to prevent development of, or alleviate to some extent, one or more of the symptoms of the condition or disorder being treated. The therapeutically effective amount may vary depending on the compound, the disorder or condition and its severity.

The terms “prevent,” “preventing,” “prevention” and grammatical variations thereof as used herein, refers to a method of partially or completely delaying or precluding the onset or recurrence of a disorder or condition and/or one or more of its attendant symptoms or barring a subject from acquiring or reacquiring a disorder or condition or reducing a subject's risk of acquiring or re-acquiring a disorder or condition or one or more of its attendant symptoms.

The phrase “selectively” or “specifically” when referring to binding to a receptor, refers to a binding reaction that is determinative of the presence of the receptor, often in a heterogeneous population of receptors and other biologics. Thus, under designated conditions, the compounds bind to a particular receptor at least two times the background and more typically more than 10 to 100 times background. Specific binding of a compound under such conditions requires a compound that is selected for its specificity for a particular receptor. For example, small organic molecules can be screened to obtain only those compounds that specifically or selectively bind to a selected receptor and not with other receptors or proteins. A variety of assay formats may be used to select compounds that are selective for a particular receptor. For example, High-throughput screening assays are routinely used to select compounds that are selective for a particular a receptor.

The term “subject” is defined herein to include animals such as mammals, including, but not limited to, primates (e.g., humans), cows, sheep, goats, horses, dogs, cats, rabbits, rats, mice and the like. In one embodiment, the subject is a human.

As used herein, “Syk” and “Syk kinase” are used interchangeably and refer to the same protein.

As used herein, “Syk inhibitor” and “Syk kinase inhibitor” are used interchangeably.

The terms “Syk inhibitor” and “Syk kinase inhibitor” refer to any agent that inhibits the catalytic activity of the Syk protein tyrosine kinase. As used herein, Syk kinase inhibitor includes small molecules.

The terms “treat,” “treating,” “treatment” and grammatical variations thereof as used herein, includes partially or completely delaying, alleviating, mitigating or reducing the intensity of one or more attendant symptoms of a disorder or condition and/or alleviating, mitigating or impeding one or more causes of a disorder or condition. Treatments according to the disclosure may be applied preventively, prophylactically, pallatively or remedially.

The disclosure provides methods and compositions for treating malaria and similar parasitic diseases by administration of a Syk kinase inhibitor. Syk kinase is one of the two known members of the Syk family (Syk and ZAP-70) non-receptor tyrosine kinases. Syk is activated upon the binding of its src homology 2 (SH2) domains to immunoreceptor tyrosine-based activation motifs (ITAM).

### I. Malaria

Malaria is caused by a parasite, Plasmodium falciparum, which is passed from one human to another by the bite of infected Anopheles mosquitoes. After infection, the parasites (called sporozoites) travel through the bloodstream to the liver, where they mature and release another form, the merozoites.

The parasites enter the bloodstream and infect red blood cells (RBCs). The parasite develops in a parasitophorous vacuole (PV) through the ring stage (0-12 hours), trophozoite stage (24-36 hours) and schizont stage (early about 36 hours; mid about 40 hours and late about 48 hours). In mature-stage parasites (greater than 24 hours), membrane bound structures appear in the RBC cytoplasm and knobby deformations are formed at the RBC membrane. After approximately 48 hours, the infected RBC ruptures, releasing the 16-32 daughter merozoites. Degradation of haemoglobin results in the deposition of crystals of haemozoin in a digestive vacuole.

In contrast to early stage parasitized RBCs, where a stable red blood cell membrane (RBCM) is necessary to protect the developing Plasmodiur, when the parasite reaches maturity, it must escape from the RBC. Thus, for this purpose, a weakened RBCM would be conducive to parasite proliferation, survival and specifically egress from infected RBCs.

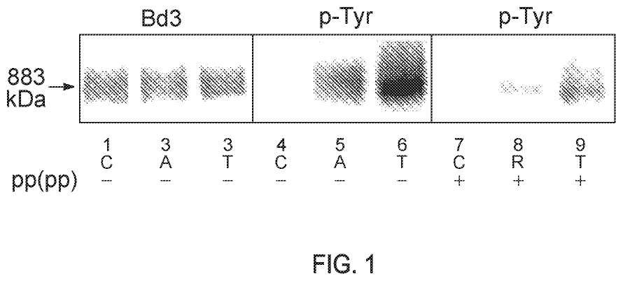

Accordingly, the inventors hypothesized that inhibition of p72 Syk-mediated phosphorylation of band 3 during the later stages of parasite maturation (trophozoite or schizont stage) might block parasite egress from the infected RBC, leaving the infected cell filled with merozoites that are unable to release into the blood. In essence, by blocking Syk phosphorylation of band 3, the band 3 will maintain a strong interaction with ankyrin and thereby stabilize the RBCM preventing parasite egress. This hypothesis is supported by four clear observations: (i) band 3 tyrosine phosphorylation peaks immediately before parasite egress from human erythrocytes (FIG. 1) [Pantaleo, Proteomics 2010, 10:3469-34 79], (ii) tyrosine phosphorylation of band 3 destabilizes the RBCM, even in healthy cells, leading to membrane fragmentation, (iii) treatment of erythrocytes with an inhibitor of p72 Syk blocks tyrosine phosphorylation of band 3 and (iv) treatment of Plasmodium-infected RBCs with a Syk kinase inhibitor prevents parasite escape from the infected erythrocytes (see Examples and figures below). Because the entrapped merozoites die if they fail to escape their host erythrocyte within a few hours, this failure to burst out of the RBC essentially terminates the malaria infection.

### II. Syk or Syk Kinase

Syk kinase plays an essential role in lymphocyte development and activation of immune cells and is best characterized for its role in B cell receptor signaling and Fe receptor mediated release of mast cell granules. Although expressed ubiquitously in hematopoietic cells, Syk is also expressed in other tissues, such as breast epithelial cells and hepatocytes.

“Syk” or “Syk kinase” refers to the 72 kDa non-receptor (cytoplasmic) spleen protein tyrosine kinase expressed in B-cells and other hematopoetic cells. Syk kinase is characterized by two consensus Src-homology 2 (SH2) domains in tandem that bind to phosphorylated immunoreceptor tyrosine-based activation motifs (“ITAMs”), a “linker” domain and a catalytic domain (for a review, see Sada et al., 2001, J. Biochem. (Tokyo) 130:177-186 and also Turner et al., 2000, Immunology Today 21:148-154 and Wong et al., 2004, Expert Opin Investig Drugs 13(7):743-62).

Syk kinase is also critical for tyrosine phosphorylation of multiple proteins that regulate important pathways leading from immunoreceptors, such as Ca²⁺ mobilization and mitogen-activated protein kinase (MAPK) cascades and degranulation. Syk kinase also plays a critical role in integrin signaling in neutrophils (see, e.g., Mocsai et al. 2002, Immunity 16:547-558).

Syk kinase includes kinases from any species of animal, including but not limited to, Homo sapiens, simian, bovine, porcine, rodent, etc., recognized as belonging to the Syk family. Specifically included are isoforms, splice variants, allelic variants, mutants, both naturally occurring and man-made. The amino acid sequences of such Syk kinases are available from GENBANK. Specific examples of mRNAs encoding different isoforms of human Syk kinase are available at GENBANK accession no. gi|21361552|ref|NM.sub.-003177.2, gi|496899|emb|Z29630.1|HSSYKPTK[496899] and gi|15030258|gb|BC011399.1|BC011399[15030258], which are incorporated herein by reference.

Syk mediated integrin signaling refers to signal transduction of cell surface integrins that occur via interaction with Syk kinase. Integrins comprise an extended family of cell surface adhesion receptors that bind extracellular matrix and cell surface ligands. Structurally, integrins are heterodimeric proteins composed of an alpha and beta chain, where each subunit has an extracellular domain, a single transmembrane domain, and a cytoplasmic domain. The α-subunit generally composed of about seven tandem repeats, where a subset of the repeats contain putative metal binding sequences of the general structure DxDxDGxxD, where x is any amino acid. Two groups of integrins can be characterized by the alpha subunits: those that contain an “A” domain and those having a proteolytic cleavage site. The β-subunit comprises a conserved region of about 200 amino acids in the extracellular domain, which is characterized by a region having structural similarity to the “A” domain of the a subunit and another region with epidermal growth factor (EGF) like repeats, similar to those found in laminin (see, e.g., Xiong et al., 2003, Blood, 102(4):1155-1159).

Integrin activity may modulate intracellular Syk, or conversely, the integrin function can be modulated via the activity of Syk. It is generally understood that in some instances, integrins require activation within the cell to bind its cognate ligands (inside-out activation). Integrins that either modulate or are modulated by Syk include, among others, beta-1-integrins (Lin et al., J Biol. Chem. 1995, 270(27):16189-97) such as .alpha_{2b1}, beta-2 integrins, and beta-3 integrins such as α_{11b}.β₃. For instance, it is believed that Syk binds directly to the integrin β₃ cytoplasmic tail through the SH2 domains. However, unlike Syk binding to ITAMs, the interaction with β₃ integrin appears independent of the phosphotyrosine binding function of the tandem SH2 domains.

### III. Syk Inhibitors/Syk Kinase Inhibitors

In one embodiment, the disclosure relates to a method of treating malaria comprising administering an effective amount of a Syk kinase inhibitor to a subject in need of treatment. In one embodiment, more than one Syk kinase inhibitor can be used. In another embodiment, two or more Syk kinase inhibitors can be used, wherein the inhibitors are administered sequentially. In another embodiment, two or more Syk kinase inhibitors can be used, wherein the inhibitors are administered concurrently or simultaneously.

In one embodiment, the disclosure relates to a method for treating malaria in a patient comprising: (1) identifying a patient in need of treatment from malaria; (2) administering to said patient a therapeutically effective amount of a Syk kinase inhibitor.

In yet another embodiment, the disclosure relates to a method for reducing the incidence of malaria comprising: identifying a subject who may be a carrier of malaria; and (2) administering to said patient a therapeutically effective amount of a Syk kinase inhibitor.

In still another embodiment, the disclosure relates to a method for inhibiting the growth of Plasmodium falciparum in a patient comprising administering an Syk kinase inhibitor in an effective amount to inhibit the growth of Plasmodium falciparum.

Any Syk kinase inhibitor or combination of Syk kinase inhibitors that achieves the desired result may be used in the compositions and methods disclosed herein. One or more than one Syk kinase inhibitor can be used.

In one embodiment, any number and any combination of Syk kinase inhibitors can be used, including but not limited to 1, 2, 3, 4, 5, 6, 7, 8, 9, 10, 11-15, 16-20, and 21-25, 26-35, 36-50, 51-100, 101-150, 151-200, and greater than 200 Syk kinase inhibitors. One or more than one mechanism of inhibition may be used including but not limited to small molecule inhibitors, shRNA, RNA interference, and small interfering RNA.

In another embodiment, any dosage or concentration of Syk kinase inhibitor that achieves the desired result may be used including but not limited to from 100 to about 2000 mg/day, from about 100 to about 1800 mg/day, from about 100 to about 1600 mg/day, from about 100 to about 1400 mg/day, from about 100 to about 1200 mg/day, from about 100 to about 1000 mg/day, from about 100 to about 800 mg/day, from about 100 to about 600 mg/day, from about 100 to about 400 mg/day, and from about 100 to about 200 mg/day.

In another embodiment, any dosage or concentration of Syk kinase inhibitor that achieves the desired result may be used including but not limited to from 400 to about 2000 mg/day, from about 400 to about 1800 mg/day, from about 400 to about 1600 mg/day, from about 400 to about 1400 mg/day, from about 400 to about 1200 mg/day, from about 400 to about 1000 mg/day, and from about 400 to about 800 mg/day.

In one embodiment, the Syk kinase inhibitor can inhibit or reduce the activity of Syk by any amount including but not limited to 1-5%, 5-10%, 10-20%, 20-30%, 30-40%, 40-50%, 50-60%, 60-70%, 70-80%, 80-90%, 90-95%, and 95-99%, 99-200%, 200-300%, 300-400%, 400-500% and greater than 500% as compared to the normal activity of Syk (without the inhibitor).

In one embodiment, the Syk kinase inhibitor can inhibit or reduce the activity of Syk from about 5% to about 20%, from about 5% to about 30%, from about 5% to about 40′, from about 5% to about 50%, from about 5% to about 60%, from about 5% to about 70%, from about 5% to about 80%, from about 5% to about 90%, and from about 5% to about 95% as compared to the normal activity of Syk without the inhibitor.

Examples of Syk kinase inhibitors include, without limitation, NVP-QAB205; purine-2-benzamine derivatives such as those described in U.S. Pat. No. 6,589,950, hereby incorporated by reference herein; pyrimidine-5-carboxamide derivatives such as those described in PCT Publication No. WO 99/31073, hereby incorporated by reference herein; 1,6-naphthyridine derivatives such as those described in U.S. Patent Publication No. 2003/0229090, hereby incorporated by reference herein; BAY 61-3606; piceatannol; 3,4-dimethyl-10-(3-aminopropyl)-9-acridone oxalate); and combinations thereof.

Additional examples of Syk kinase inhibitors include, without limitation, compounds and derivatives disclosed in U.S. Patent Application Publication No. 20120130073; compounds and derivatives disclosed in U.S. Patent Application Publication No. 20100316649; compounds and derivatives disclosed in U.S. Pat. Nos. 8,057,815; 8,258,144; 8,227,455; 8,138,339; 8,063,058; 8,012,959; 7,842,712; 7,803,801; 7,705,004; 7,678,911; 7,547,794; 7,501,410; 7,449,456; 7,446,199, 7,321,041; 7,304,071; 7,276,502; 7,262,200, 7,173,015; 6,911,443; and 6,797,706; all of which are hereby incorporated by reference in their entirety.

ER-27319 (3,4-dimethyl-10-(3-aminopropyl)-9-acridone oxalate) can be used to inhibit Syk. Various concentrations of piceatannol (3,4,3′5′-tetrahydroxy-trans-stilbene) can also be used as a Syk kinase inhibitor.

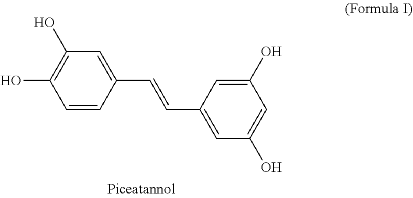 [SMILES: Oc1cc(O)cc(/C=C/c2ccc(O)c(O)c2)c1]

In addition to the inhibitors mentioned above, WO 0109134 discloses purine derivatives as inhibitors of SYK kinase. WO 0147922 describes substituted azaindoles useful in the treatment of disease states capable of being modulated by the inhibition of protein kinases, in particular SYK kinase. WO 9818782 describes inhibitors of ZAP70 that are also reported to inhibit SYK.

Recently, R406 (Rigel Pharmaceuticals) was reported to inhibit ITAM signaling in response to various stimuli, including Fe.epsilon.R1 and BCR induced Syk activation (Braselmann, Taylor et al. J Pharmacol Exp Ther 319(3): 998-1008 (2006). Interestingly, this ATP-competitive inhibitor of Syk was also active against Flt3, cKit, and JAK kinases, but not against Src kinases (Braselmann, Taylor et al. 2006). Activating mutations to Flt3 are associated with AML and inhibition of this kinase is currently under clinical development (Burnett and Knapper Hematology Am Soc Hematol Educ Program 2007: 429-34 (2007). Over-activation of the tyrosine kinase cKit is also associated with hematologic malignancies, and a target for cancer therapy (Heinrich, Griffith et al. Blood 96(3): 925-32 (2000). Similarly, JAK3 signaling is implicated in leukemias and lymphomas, and is currently exploited as a potential therapeutic target (Heinrich, Griffith et al. 2000). Importantly, the multi-kinase inhibitory activity of R406 attenuates BCR signaling in lymphoma cell lines and primary human lymphoma samples, resulting in apoptosis of the former (Chen, Monti et al. Blood 111(4): 2230-7 (2008). Further, a phase II clinical trial reported favorable results by this compound in refractory NHL and chronic lymphocytic leukemia (Friedberg J W et al, Blood 2008; 112(11), Abstract 3). The R406 data suggest that inhibition of kinases that mediate survival signaling in lymphocytes is clinically beneficial.

In one embodiment, R406 and derivatives thereof can be used to treat malaria. In another embodiment, the disclosure relates to a method of treating malaria comprising administering to a subject in need of such treatment a therapeutically effective amount of the compound of Formula (II) or a pharmaceutically acceptable salt, a pharmaceutical composition or a medicament thereof.

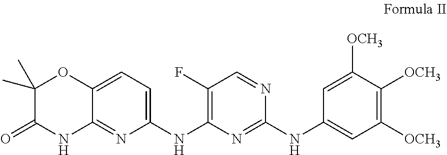 [SMILES: COc1cc(Nc2ncc(F)c(Nc3ccc4c(n3)NC(=O)C(C)(C)O4)n2)cc(OC)c1OC]

In another embodiment, R788, Fostamatinib disodium, (Rigel Pharmaceuticals) and derivatives thereof can be used to treat malaria. In another embodiment, the disclosure relates to a method of treating malaria comprising administering to a subject in need of such treatment a therapeutically effective amount of the compound of Formula (III) or a pharmaceutically acceptable salt, a pharmaceutical composition or a medicament thereof.

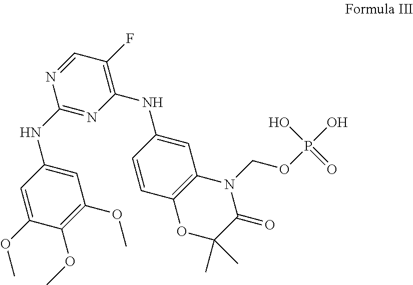 [SMILES: COc1cc(Nc2ncc(F)c(Nc3ccc4c(c3)N(COP(=O)(O)O)C(=O)C(C)(C)O4)n2)cc(OC)c1OC]

Fostamatinib is an experimental drug candidate for the treatment of a variety of diseases. It is in Phase III clinical trials for rheumatoid arthritis and Phase II trials for autoimmune thrombocytopenia and lymphoma. The oral drug is used as its disodium salt and it is a prodrug of the active compound tamatinib (R-406), which is an inhibitor of the enzyme spleen tyrosine kinase (Syk).

In one embodiment, compounds useful for treating malaria include compounds of the structure below:

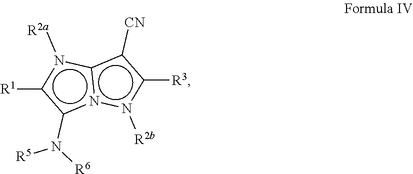

wherein R¹ is selected from aryl, substituted aryl, heteroaryl, substituted heteroaryl, cycloalkyl, substituted cycloalkyl, cycloalkenyl, substituted cycloalkenyl, heterocyclyl, substituted heterocyclyl, aralkyl, heteroaralkyl, hydrogen, alkyl, substituted alkyl, alkenyl, substituted alkenyl, alkynyl, substituted alkynyl, acyl, acylamino, and acyloxy;

R^{2a} and R^{2b} are independently selected from hydrogen, alkyl, substituted alkyl, acyl, acylamino, acyloxy, —SO-alkyl, —SO-aryl, —SO-heteroaryl, —SO₂-alkyl, —SO₂-aryl, —SO₂-heteroaryl, aryl, substituted aryl, heteroaryl, heterocyclyl, aralkyl, and heteroaralkyl; and wherein either R^{2a} or R^{2b} is present;

R³ is selected from hydrogen, alkyl, substituted alkyl, alkenyl, substituted alkenyl, halo, nitro, cyano, hydroxy, alkoxy, carboxyl, acyl, acylamino, aminoacyl, acyloxy, oxyacyl, amino, substituted amino, aryl, substituted aryl, heteroaryl, and substituted heteroaryl;

R⁵ is selected from hydrogen, alkyl, and substituted alkyl; and

R⁶ is selected from hydrogen, alkyl, substituted alkyl, alkenyl, substituted alkenyl, alkynyl, substituted alkynyl, acyl, acylamino, acyloxy, cycloalkyl, substituted cycloalkyl, cycloalkenyl, substituted cycloalkenyl, aralkyl, heteroaralkyl, aryl, substituted aryl, heteroaryl, substituted heteroaryl, heterocyclyl, and substituted heterocyclyl; or a salt or stereoisomer thereof.

In yet another embodiment, compounds useful for treating malaria include compounds of the structure below:

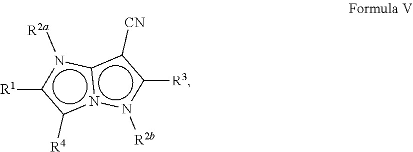

wherein R¹ is selected from aryl, substituted aryl, heteroaryl, substituted heteroaryl, cycloalkyl, substituted cycloalkyl, cycloalkenyl, substituted cycloalkenyl, heterocyclyl, substituted heterocyclyl, aralkyl, heteroaralkyl, hydrogen, alkyl, substituted alkyl, alkenyl, substituted alkenyl, alkynyl, substituted alkynyl, acyl, acylamino, and acyloxy;

R^{2a} and R^{2b} are independently selected from hydrogen, alkyl, substituted alkyl, acyl, acylamino, acyloxy, —SO-alkyl, —SO-aryl, —SO-heteroaryl, —SO₂-alkyl, —SO₂-aryl, —SO₂-heteroaryl, aryl, substituted aryl, heteroaryl, heterocyclyl, aralkyl, and heteroaralkyl, and wherein either R² or R^{2b} is present;

R³ is selected from hydrogen, alkyl, substituted alkyl, alkenyl, substituted alkenyl, halo, nitro, cyano, hydroxy, alkoxy, carboxyl, acyl, acylamino, aminoacyl, acyloxy, oxyacyl, amino, substituted amino, aryl, substituted aryl, heteroaryl, and substituted heteroaryl;

R⁴ is selected from hydrogen, alkyl, substituted alkyl, amino, or —NR⁵R⁶;

R⁵ is selected from hydrogen, alkyl, and substituted alkyl; and

R⁶ is selected from aryl, substituted aryl, heteroaryl, substituted heteroaryl, cycloalkyl, substituted cycloalkyl, cycloalkenyl, substituted cycloalkenyl, heterocyclyl, substituted heterocyclyl, aralkyl, heteroaralkyl, hydrogen, alkyl, substituted alkyl, alkenyl, substituted alkenyl, alkynyl, substituted alkynyl, acyl, acylamino, and acyloxy; or a salt or stereoisomer thereof.

In another embodiment, oxindoles, including but not limited to compounds listed in Table I, can be used to inhibit Syk activity.

TABLE I
Comparison of the physiochemical properties and inhibitory
activities of oxindoles in kinase and whole cell based assays.
Formula VI

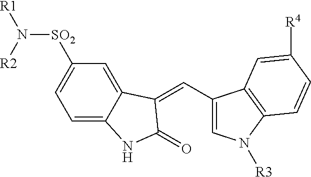

| | | | | | | | Solubility | |
|---|---|---|---|---|---|---|---|---|
| Compd | R1 | R2 | R3 | R4 | IC₅₀ (nM) | EC₅₀ (nM) | (mg L) | PSA |
| 1 | H | H | H | H | 128 | | | 121 |
| 2 | H | H | Me | H | 14 | 313 | | 108 |
| 3 | H | H | H | OMe | 28 | >10,000 | | 162 |
| 31 | H | H | Me | OMe | 5 | 1400 | <0.1 | 117 |
| 6 | Me | Me | Me | OMe | Inactive @ | >10,000 | <0.1 | 94 |
| | | | | | 80 μM | | | |
| 7 | H | Me | Me | OMe | 20 | 11n | <0.1 | 105 |
| 8 | H | Et | Me | OMe | 100 | | | 104 |
| 9 | H | ′Bu | Me | OMe | 937 | | | 101 |
| 10 | H | p- | Me | OMe | 20% @ 4 | | | 116 |
| | | MeOPhCH₂ | | | μM | | | |
| 11 | H | CH₂COOH | Me | OMe | 658 | 1940 | 30 | 148 |
| 12 | H | CH₂CH₂C(—O)NH₂ | Me | OMe | 60 | >10,000 | 8 | 151 |
| 13 | H | CH₂C(—O)NH₂ | Me | OMe | 98 | >10,000 | 1 | 152 |
| 14 | H | H | CH₂COOH piperidine salt | OMe | 850 | | 70 | 163 |
| 15 | H | H | (CH₂)₃COOH | OMe | 14 | | | 163 |
| 16 | H | H | (CH₂)₄COOH | OMe | 11 | 20,700 | <0.1 | 163 |
| 17 | H | H | 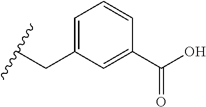 [SMILES: *Cc1cccc(C(=O)O)c1] | OMe | 27 | | | 149 |
| 18 | H | H | (CH₂)₃OH | OMe | 32 | >10,000 | <0.1 | 142 |
| 19 | H | H | (CH₂)ₜCONH₂ | OMe | 12 | >10,000 | 5 | 164 |
| 20 | H | H | CH₂COO′Bu | OMe | 1000 | | | 140 |
| 21 | H | H | (CH₂)₃COOEt | OMe | 204 | | | 147 |
| 22 | H | H | (CH₂)₄COOEt | OMe | 678 | 6960 | <0.1 | 147 |
| 23 | H | H | (CH₂)₅COOEt | OMe | 465 | 6300 | <0.1 | 147 |
| 24 | H | H | (CH₂)₃CO-morpholino | H | 133 | 5600 | <0.1 | 153 |
| 25 | H | H | (CH₂)₃CO-piperidine | H | 616 | >30,000 | 10 | 156 |

Adopted from Lai et al, Biorganic and Medicinal Chemistry Letters, 1:3111-3114 (2003).

In another embodiment, compounds useful in the methods of the disclosure include but are not limited to:

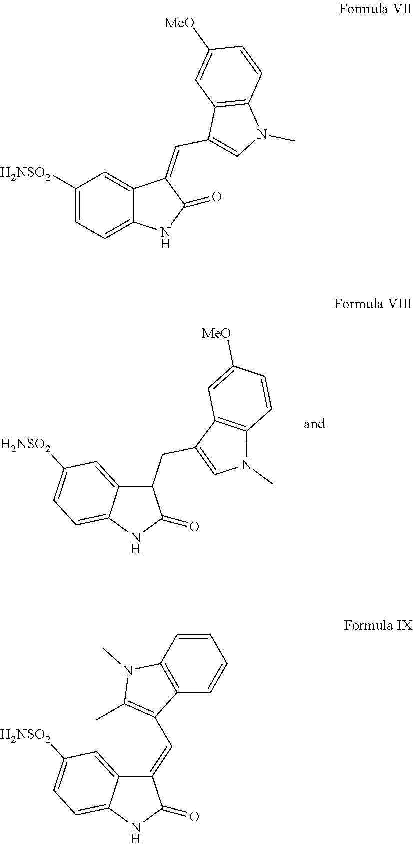

The compounds embodied by formulas VII, VIII, and IX are described in Lai et al. (Biorganic and Medicinal Chemistry Letters 13:3111-3114, 2003), which is entitled “Potent Small Molecule Inhibitors of Spleen Tyrosine Kinase (Syk).”

In yet another embodiment, the Syk kinase inhibitor can have the chemical designation of: 3,3′-[(5-Fluoro-2,4-pyrimidinediyl)diimino]bis-phenol (R 12).

A. Pyrimidine Derivatives

In one embodiment, the Syk kinase inhibitor is a pyrimidine derivative. In one embodiment, the Syk kinase inhibitor is a N-phenyl-2-pyrimidine-amine derivative of formula X:

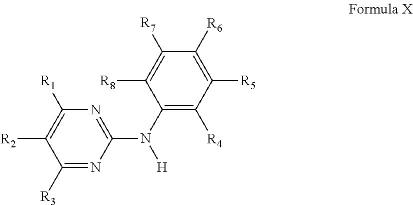

wherein

R₁ is 4-pyrazinyl, 1-methyl-1H-pyrrolyl, amino- or amino-lower alkyl-substituted phenyl wherein the amino group in each case is free, alkylated or acylated, 1H-indolyl or 1H-imidazolyl bonded at a five-membered ring carbon atom, or unsubstituted or lower alkyl-substituted pyridyl bonded at a ring carbon atom and unsubstituted or substituted at the nitrogen atom by oxygen;
R₂ and R₃ are each independently of the other hydrogen or lower alkyl, one or two of the radicals R₄, R₅, R₆, R₇ and R₈ are each nitro, fluoro-substituted lower alkoxy or a radical of formula XI—

N(R₉)—C(═X)—(Y)ₙ—R₁₀ (Formula XI)

wherein
R₉ is hydrogen or lower alkyl,
X is oxo, thio, imino, N-lower alkyl-imino, hydroximino or O-lower alkyl-hydroximino,
Y is oxygen or the group NH,

### N is 0 or 1 and

R₁₀ is an aliphatic hydrocarbon radical having at least 5 carbon atoms, or an aromatic, aromatic-aliphatic, cycloaliphatic, cycloaliphatic-aliphatic, heterocyclic or heterocyclicaliphatic radical, and the remaining radicals R₄, R₅, R₆, R₇ and R₈ are each independently of the others each independently of the others hydrogen, lower alkyl that is unsubstituted or substituted by free or alkylated amino, piperazinyl, piperidinyl, pyrrolidinyl or by morpholinyl, or lower alkanoyl, trifluoromethyl, free, etherified or esterifed hydroxy, free, alkylated or acylated amino or free or esterified carboxy, or a salt of such a compound having at least one salt-forming group.

In one embodiment, R₁₀ is a phenyl or naphthyl radical each of which is unsubstituted or substituted by cyano, trifluoromethyl, hydroxy, lower alkoxy, lower alkanoyloxy, halogen, amino, lower alkylamino, di-lower alkylamino, lower alkanoylamino, benzoylamino, carboxy, lower alkoxycarbonyl or by unsubstituted or substituted lower alkyl, or phenyl-lower alkyl wherein the phenyl radical is unsubstituted or substituted as indicated above, a cycloalkyl or cycloalkenyl radical having up to 30 carbon atoms, cycloalkyl-lower alkyl or cycloalkenyl-lower alkyl each having up to 30 carbon atoms in the cycloalkyl or cycloalkenyl moiety, a monocyclic radical having 5 or 6 ring members and 1-3 ring atoms selected from nitrogen, oxygen and sulfur, to which radical one or two benzene radicals may be fused, or lower alkyl substituted by such a monocyclic radical.

In one embodiment, the Syk kinase inhibitor is imatinib mesylate (Gleevec). Imatinib mesylate is designated chemically as 4-[(4-Methyl-1-piperazinyl)methyl]-N-[4-methyl-3-[[4-(3-pyridinyl)-2-pyrimidinyl]amino]-phenyl]benzamide methanesulfonate and its structural formula is

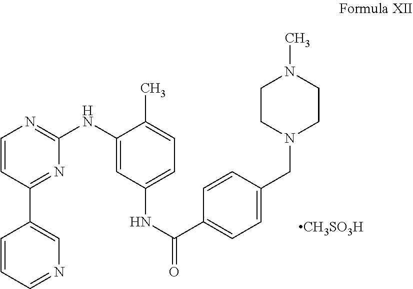

In one embodiment, the Syk kinase inhibitor has the structural formula of:

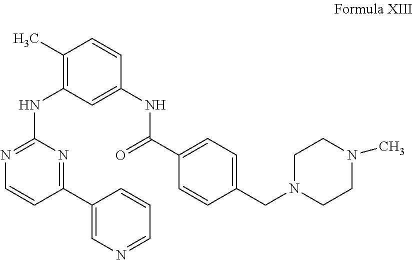 [SMILES: Cc1ccc(NC(=O)c2ccc(CN3CCN(C)CC3)cc2)cc1Nc1nccc(-c2cccnc2)n1]

In one embodiment, the disclosure relates to a method of treating a parasitic disease comprising administering imatinib mesylate to a subject in need of treatment. In another embodiment, the method comprises identifying a subject in need of treatment for a parasitic disease. In another embodiment, the parasitic disease is malaria.

In one embodiment, imatinib mesylate is administered with one or more Syk kinase inhibitors. In still another embodiment, imatinib mesylate is administered with one or more anti-malarial drugs.

In yet another embodiment, imatinib mesylate is administered from about 200 to about 1200 mg/day. In another embodiment, imatinib mesylate is administered from about 400 to about 1200 mg/day. In another embodiment, imatinib mesylate is administered from about 400 to about 1000 mg/day. In another embodiment, imatinib mesylate is administered from about 400 to about 800 mg/day. In another embodiment, imatinib mesylate is administered from about 400 to about 600 mg/day.

In one embodiment, imatinib mesylate is administered at about 800 mg/day.

Imatinib mesylate has a molecular formula is C₂₉H₃₁N₇O.CH₄SO₃ and its molecular weight is 589.7. Imatinib mesylate is soluble in aqueous buffers </=pH 5.5 but is very slightly soluble to insoluble in neutral/alkaline aqueous buffers. In non-aqueous solvents, the drug substance is freely soluble to very slightly soluble in dimethyl sulfoxide, methanol and ethanol, but is insoluble in n-octanol, acetone and acetonitrile.

Imatinib mesylate is a protein-tyrosine kinase inhibitor that inhibits the bcr-abl tyrosine kinase, the constitutive abnormal tyrosine kinase created by the Philadelphia chromosome abnormality in chronic myeloid leukemia (CML). It inhibits proliferation and induces apoptosis in bcr-abl positive cell lines as well as fresh leukemic cells from Philadelphia chromosome positive chronic myeloid leukemia. In colony formation assays using ex vivo peripheral blood and bone marrow samples, imatinib shows inhibition of bcr-abl positive colonies from CML patients.

In vivo, imatinib inhibits tumor growth of bcr-abl transfected murine myeloid cells as well as bcr-abl positive leukemia lines derived from CML patients in blast crisis.

Imatinib is also an inhibitor of the receptor tyrosine kinases for platelet-derived growth factor (PDGF) and stem cell factor (SCF), c-kit, and inhibits PDGF- and SCF-mediated cellular events. In vitro, imatinib inhibits proliferation and induces apoptosis in gastrointestinal stromal tumor (GIST) cells, which express an activating c-kit mutation.

The pharmacokinetics of Gleevec® (imatinib mesylate) have been evaluated in studies in healthy subjects and in population pharmacokinetic studies in over 900 patients. Imatinib is well absorbed after oral administration with Cₘₐₓ achieved within 2-4 hours post-dose. Mean absolute bioavailability is 98%.

Following oral administration in healthy volunteers, the elimination half-lives of imatinib and its major active metabolite, the N-desmethyl derivative, are approximately 18 and 40 hours, respectively. Mean imatinib AUC increases proportionally with increasing doses ranging from 25 mg-1,000 mg. There is no significant change in the pharmacokinetics of imatinib on repeated dosing, and accumulation is 1.5- to 2.5-fold at steady state when Gleevec® is dosed once daily. At clinically relevant concentrations of imatinib, binding to plasma proteins in in vitro experiments is approximately 95%, mostly to albumin and (alpha)₁-acid glycoprotein. The pharmacokinetics of are similar in CML and GIST patients.

CYP3A4 is the major enzyme responsible for metabolism of imatinib. Other cytochrome P450 enzymes, such as CYP1A2, CYP2D6, CYP2C9, and CYP2C19, play a minor role in its metabolism. The main circulating active metabolite in humans is the N-demethylated piperazine derivative, formed predominantly by CYP3A4. It shows in vitro potency similar to the parent imatinib. The plasma AUC for this metabolite is about 15% of the AUC for imatinib. The plasma protein binding of the N-demethylated metabolite CGP71588 is similar to that of the parent compound

Elimination is predominately in the feces, mostly as metabolites. Based on the recovery of compound(s) after an oral ¹⁴C-labeled dose of imatinib, approximately 81% of the dose was eliminated within 7 days, in feces (68% of dose) and urine (13% of dose). Unchanged imatinib accounted for 25% of the dose (5% urine, 20% feces), the remainder being metabolites.

Typically, clearance of imatinib in a 50-year-old patient weighing 50 kg is expected to be 8 L/h, while for a 50-year-old patient weighing 100 kg the clearance will increase to 14 L/h. However, the inter-patient variability of 40% in clearance does not warrant initial dose adjustment based on body weight and/or age but indicates the need for close monitoring.

B. Syk Kinase Inhibitor II

In one embodiment, the disclosure relates to a method of treating a parasitic disease comprising administering Syk kinase inhibitor II to a subject in need of treatment. In another embodiment, the method comprises identifying a subject in need of treatment for a parasitic disease. In another embodiment, the parasitic disease is malaria.

In one embodiment, Syk kinase inhibitor II is administered with one or more Syk kinase inhibitors. In still another embodiment, Syk kinase inhibitor II is administered with one or more anti-malarial drugs.

In yet another embodiment, Syk kinase inhibitor II is administered from about 200 to about 1200 mg/day. In another embodiment, Syk kinase inhibitor II is administered from about 400 to about 1200 mg/day or from about 400 to about 1000 mg/day or from about 400 to about 800 mg/day or from about 400 to about 600 mg/day.

In one embodiment, Syk kinase inhibitor II is administered at about 800 mg/day.

In one embodiment, Syk kinase inhibitor II has the following formula:

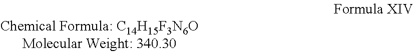

In another embodiment, Syk kinase inhibitor II is designated chemically as 2-(2-Aminoethylamino)-4-(3-trifluoromethylanilino)-pyrimidine-5-carboxamide

C. Syk Kinase Inhibitor IV

In one embodiment, the disclosure relates to a method of treating a parasitic disease comprising administering Syk kinase inhibitor IV to a subject in need of treatment. In another embodiment, the method comprises identifying a subject in need of treatment for a parasitic disease. In another embodiment, the parasitic disease is malaria.

In one embodiment, Syk kinase inhibitor IV is administered with one or more Syk kinase inhibitors. In still another embodiment, Syk kinase inhibitor IV is administered with one or more anti-malarial drugs.

In yet another embodiment, Syk kinase inhibitor IV is administered from about 200 to about 1200 mg/day. In another embodiment, Syk kinase inhibitor IV is administered from about 400 to about 1200 mg/day or from about 400 to about 1000 mg/day or from about 400 to about 800 mg/day or from about 400 to about 600 mg/day.

In one embodiment, Syk kinase inhibitor IV is administered at about 800 mg/day.

In one embodiment, Syk kinase inhibitor IV has the following formula:

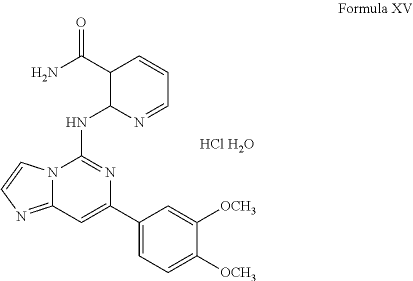 [SMILES: COc1ccc(-c2cc3nccn3c(NC3N=CC=CC3C(N)=O)n2)cc1OC ; "HCl H2O"]

### IV. Inhibition of Syk Kinase Expression

The disclosure relates to RNA molecules that target Syk kinase mRNA. For example, the disclosure relates to RNA molecules from about 19, 20 or 21 to about 23 nucleotides in length that direct cleavage and/or degradation of Syk kinase mRNA.

In one embodiment, the disclosure relates to the use of siRNA molecules, double stranded RNA molecules typically comprising two 20-23 nucleotide (nt) strands. SiRNAs suitable for use in the disclosure can be produced using any of a variety of approaches. The siRNA can be prepared in vitro and then introduced directly into cells (for example, by transfection). Alternatively, intracellular expression can be effected by transfecting into cells constructs (e.g., DNA-based vectors or cassettes) that express siRNA within cells.

More specifically, siRNA suitable for use in the disclosure can be prepared, for example, via chemical synthesis, in vitro transcription, enzymatic digestion of a longer dsRNA using an RNase III enzyme such as Dicer or RNase III, expression in cells from an siRNA expression plasmid or viral vector, or expression in cells from a PCR-derived siRNA expression cassette. Detailed descriptions of these various approaches are readily available and can be found, for example, at http://www.ambion.com/techlib/tn/103/2.html, www.bdbiosciences.com, www.oligoengine.com, www.genetherapysystems.com, www.dharmacon.com, http://www.mpibpc.gwdg.de/abteilungen/100/105/sirna.html, and/or in the references cited therein (which references are also incorporated herein by reference). (See also Sui et al, Proc Natl Acad Sci USA 99.5515-20 (2002), Brummelkamp et al, Science 296:550-3 (2002), Paul et al, Nature Biotechnology 20:505-8 (2002), Lee et al, Nature Biotechnology 20: 500-5 (2002), Castanotto et al, RNA 8: 1454-60 (2002) and US Appln. 20030108923.)

As indicated above, siRNA suitable for use in the disclosure can be prepared chemically. Advantageously, 2′ hydroxyls are protected during the synthetic process against degradation using, for example, acid labile orthoester protecting groups (see Scaringe et al, J. Am. Chem. Soc. 120:11820 (1998) and www.dharmacon.com (e.g., the ACE technology described therein)). The RNA oligomers can be simultaneously 2′ deprotected and annealed prior to use.

In chemically synthesized siRNA, at least one strand of the double stranded molecule can have a 3′ overhang from about 1 to about 6 nucleotides (e.g., pyrimidine and/or purine nucleotides) in length. Preferably, the 3′ overhang is from about 1 to about 5 nucleotides (e.g., thymidines or uridines), more preferably from about 1 to about 4 nucleotides and most preferably 2 or 3 nucleotides in length. Advantageously, each strand has an overhang. The length of the overhangs can be the same or different for each strand. Typically, both strands have overhangs of the same length. In a particular embodiment, the RNA of the present disclosure comprises 21 or 22 nucleotide strands that are paired and that have overhangs of from about 1 to about 3, particularly, about 2, nucleotides on the 3′ ends of both of the RNA strands.

As indicated above, siRNAs suitable for use in the disclosure can be prepared by enzymatic digestion of a longer dsRNA using an RNase III type enzyme (e.g., Dicer). (See references and web sites cited above.) For example, a commercially available Dicer siRNA generation kit can be used that permits generation of large numbers of siRNAs from full length target genes (Gene Therapy Systems, Inc, MV062603). SiRNA can be produced from target DNA and T7 RNA polymerase promoter sequences using PCR based cloning. Following RNA transcription from the target sequence, recombinant Dicer can cleave the transcribed RNAi into 22 by siRNAs

Also as indicated above, siRNA molecules suitable for use in the present disclosure can also be recombinantly produced using methods known in the art. (See references and web sites cited above.) Recombinant technology permits in vivo transcription of siRNAs in mammalian cell. In accordance with this approach, vectors can be used that contain, for example, RNA polymerase III or U6 promoter sequences. Such vectors (including viral vectors and plasmid vectors (such as pSIREN)) can be used as expression vectors or as shuttle vectors in conjunction with viral systems (e.g., adenoviral or retroviral systems) to introduce siRNA into mammalian cells. Vectors can be engineered to express sense and anti-sense strands of siRNAs that anneal in vivo to produce functional siRNAs. Alternatively, hairpin RNA can be expressed by inserting into a vector the sense strand (e.g., about 20 nt) of the target, followed by a short spacer (e.g., about 4 to about 10 nt), then the antisense strand of the target (e.g., about 20 nt) and, for example, about 5-6 T's as transcription terminator. The resulting RNA transcript folds back to form a stem-loop structure comprising, for example, about a 20 by stem and about a 10 nt loop with 2-3 U's at the 3′ end. (See also Paddison et al (Proc. Natl. Acad. Sci. 99:1443-1448 (2002).) Constructs suitable for use in effecting in vivo production (including selection of vectors and promoters) can be readily designed by one skilled in the art and will vary, for example, with the cell/tissue target and the effect sought. 1001851 dsRNA can be used in the methods of the disclosure provided it has sufficient homology to the targeted Syk kinase mRNA. SiRNA duplexes can be designed, for example, by searching Syk kinase cDNA for the target motif “AA(N)₁₉”, wherein N is any nucleotide, motifs with approximately 30% to 70% G/C content being preferred, those of about 50% G/C content being more preferred. The sense strand of the siRNA duplex can correspond to nucleotides 3 to 21 of the selected AA(N)₁₉ motif. The antisense strand of the siRNA duplex can have a sequence complementary to nucleotides 1 to 21 of the selected AA(N).sub.19 motif. Further design details are provided at http://www.mpibpc.gwdg.de/abteilungen/100/105/sirna.html.

Preferred target sequences include sequences unique to Syk kinase mRNA. For example, target sequences can be selected from sequences between the two SH2 domains of Syk kinase or between the second SH2 domain and the kinase domain. Representative targets include, but are not limited to, the sequences recited in Table II.

TABLE II
Target Sequences to Syk kinase

| | | Identified homologies |
|---|---|---|
| *Sequence | % GC | of 16-18/19 nucleotides |
| AATATGTGAAGCAGACATGGA | 42 | mitochondrial |
| | | ribosomal prot15 |
| AATCAAATCATACTCCTTCCC | 42 | |
| AAGAGAGTACTGTGTCATTCA | 42 | |
| AAGGAAAACCTCATCAGGGAA | 47 | inositolhexaphosphate |
| | | kinase, β globin |
| | | on Chri11 |
| AATCATACTCCTTCCCAAAGC | 47 | |
| AATTTTGGAGGCCGTCCACAA | 53 | Oxytokinase |
| AAGACTGGGCCCTTTGAGGAT | 58 | |
| AAGCAGACATGGAACCTGCAG | 58 | histamine receptor H3, |
| | | GTP binding protein |
| AACTTCCAGGTTCCCATCCTG | 58 | |
| AAGCCTGGCCACAGAAAGTCC | 63 | |
| AAGCCCTACCCATGGACACAG | 63 | |
| AACCTGCAGGGTCAGGCTCTG | 68 | |
| AAGGGGTGCAGCCCAAGACTG | 68 | γ glutamyl |
| | | transferase, |
| | | rb prot L27a |
| AACTTGCACCCTGGGCTGCAG | 68 | calcium channel α1E |
| | | subunit |
| AAGTCCTCCCCTGCCCAAGGG | 74 | NADH; ubiquinone |
| | | oxidoreductase |
| | | MLRQ subunit |
| AAGGCCCCCAGAGAGAAGCCC | 74 | |
| AATCTCAAGAATCAAATCATA | 26 | |
| AATGTTAATTTTGGAGGCCGT | 42 | |
| AATCCGTATGAGCCAGAACTT | 47 | |
| AATCGGCACACAGGGAAATGT | 53 | |
| AACCGGCAAGAGAGTACTGTG | 58 | |
| AAGGAGGTTTACCTGGACCGA | 58 | |

### V. Salts, Hydrates, and Prodrugs of Syk Kinase Inhibitors

It should be recognized that the Syk kinase inhibitors disclosed herein may be present and optionally administered in the form of salts, hydrates and prodrugs that are converted in vivo into the Syk kinase inhibitors disclosed herein. For example, it is within the scope of the disclosure to convert the Syk kinase inhibitors of the disclosure into and use them in the form of their pharmaceutically acceptable salts derived from various organic and inorganic acids and bases in accordance with procedures well known in the art.

When Syk kinase inhibitors of the disclosure possess a free base form, the Syk kinase inhibitors can be prepared as a pharmaceutically acceptable acid addition salt by reacting the free base form of the compound with a pharmaceutically acceptable inorganic or organic acid, e.g., hydrohalides such as hydrochloride, hydrobromide, hydroiodide; other mineral acids and their corresponding salts such as sulfate, nitrate, phosphate, etc.; and alkyl and monoarylsulfonates such as ethanesulfonate, toluenesulfonate and benzenesulfonate; and other organic acids and their corresponding salts such as acetate, tartrate, maleate, succinate, citrate, benzoate, salicylate and ascorbate. Further acid addition salts include, but are not limited to: adipate, alginate, arginate, aspartate, bisulfate, bisulfite, bromide, butyrate, camphorate, camphorsulfonate, caprylate, chloride, chlorobenzoate, cyclopentanepropionate, digluconate, dihydrogenphosphate, dinitrobenzoate, dodecylsulfate, fumarate, galactarate (from mucic acid), galacturonate, glucoheptonate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, iso-butyrate, lactate, lactobionate, malate, malonate, mandelate, metaphosphate, methanesulfonate, methylbenzoate, monohydrogenphosphate, 2-naphthalenesulfonate, nicotinate, nitrate, oxalate, oleate, pamoate, pectinate, persulfate, phenylacetate, 3-phenylpropionate, phosphate, phosphonate and phthalate. It should be recognized that the free base forms will typically differ from their respective salt forms somewhat in physical properties such as solubility in polar solvents, but otherwise the salts are equivalent to their respective free base forms for the purposes of the disclosure.

When the Syk kinase inhibitors disclosed herein possess a free acid form, a pharmaceutically acceptable base addition salt can be prepared by reacting the free acid form of the compound with a pharmaceutically acceptable inorganic or organic base. Examples of such bases are alkali metal hydroxides including potassium, sodium and lithium hydroxides; alkaline earth metal hydroxides such as barium and calcium hydroxides; alkali metal alkoxides, e.g., potassium ethanolate and sodium propanolate; and various organic bases such as ammonium hydroxide, piperidine, diethanolamine and N-methylglutamine. Also included are the aluminum salts of the Syk kinase inhibitors disclosed herein. Further base salts include, but are not limited to: copper, ferric, ferrous, lithium, magnesium, manganic, manganous, potassium, sodium and zinc salts. Organic base salts include, but are not limited to, salts of primary, secondary and tertiary amines, substituted amines including naturally occurring substituted amines, cyclic amines and basic ion exchange resins, e.g., arginine, betaine, caffeine, chloroprocaine, choline, N,N′-dibenzylethylenediamine (benzathine), dicyclohexylamine, diethanolamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris-(hydroxymethyl)-methylamine (tromethamine). It should be recognized that the free acid forms will typically differ from their respective salt forms somewhat in physical properties such as solubility in polar solvents, but otherwise the salts are equivalent to their respective free acid forms for the purposes of the disclosure.

Syk kinase inhibitors disclosed herein that comprise basic nitrogen-containing groups may be quaternized with such agents as (C₁₋₄) alkyl halides, e.g., methyl, ethyl, iso-propyl and tert-butyl chlorides, bromides and iodides; di (C₁₋₄) alkyl sulfates, e.g., dimethyl, diethyl and diamyl sulfates; (C₁₀₋₁₈) alkyl halides, e.g., decyl, dodecyl, lauryl, myristyl and stearyl chlorides, bromides and iodides; and aryl (C₁₋₄) alkyl halides, e.g., benzyl chloride and phenethyl bromide. Such salts permit the preparation of both water-soluble and oil-soluble compounds of the disclosure.

N-oxides of Syk kinase inhibitors disclosed herein can be prepared by methods known to those of ordinary skill in the art. For example, N-oxides can be prepared by treating an unoxidized form of the small molecule with an oxidizing agent (e.g., trifluoroperacetic acid, permaleic acid, perbenzoic acid, peracetic acid, metachloroperoxybenzoic acid, or the like) in a suitable inert organic solvent (e.g., a halogenated hydrocarbon such as dichloromethane) at approximately 0° C. Alternatively, the N-oxides of the compounds can be prepared from the N-oxide of an appropriate starting material.

Prodrug derivatives of Syk kinase inhibitors can be prepared by modifying substituents of Syk kinase inhibitors disclosed herein that are then converted to a different substituent. It is noted that in many instances, the prodrugs themselves also fall within the scope of the range of compounds according to the disclosure. For example, prodrugs can be prepared by reacting a compound with a carbamylating agent (e.g., 1,1-acyloxyalkylcarbonochloridate, para-nitrophenyl carbonate, or the like) or an acylating agent. Further examples of methods of making prodrugs are described in Saulnier et al. (1994), Bioorganic and Medicinal Chemistry Letters, Vol. 4, p. 1985.

Protected derivatives of Syk kinase inhibitors of the disclosure can also be made. Examples of techniques applicable to the creation of protecting groups and their removal can be found in T. W. Greene, Protecting Groups in Organic Synthesis, 3.sup.rd edition, John Wiley & Sons, Inc. 1999.

Syk kinase inhibitors of the disclosure may also be conveniently prepared, or formed as solvates (e.g., hydrates). Hydrates of Syk kinase inhibitors of the disclosure may be conveniently prepared by recrystallization from an aqueous/organic solvent mixture, using organic solvents such as dioxin, tetrahydrofuran or methanol.

A “pharmaceutically acceptable salt”, as used herein, is intended to encompass any small molecule according to the disclosure that is utilized in the form of a salt thereof, especially where the salt confers on the compound improved pharmacokinetic properties as compared to the free form of compound or a different salt form of the compound. A pharmaceutically acceptable salt, as used herein, includes salts present in vivo.

The pharmaceutically acceptable salt form may also initially confer desirable pharmacokinetic properties on the compound that it did not previously possess, and may even positively affect the pharmacodynamics of the compound with respect to its therapeutic activity in the body. An example of a pharmacokinetic property that may be favorably affected is the manner in which the compound is transported across cell membranes, which in turn may directly and positively affect the absorption, distribution, biotransformation and excretion of the compound. While the route of administration of the pharmaceutical composition is important, and various anatomical, physiological and pathological factors can critically affect bioavailability, the solubility of the compound is usually dependent upon the character of the particular salt form thereof, which it utilized. One of skill in the art will appreciate that an aqueous solution of the compound will provide the most rapid absorption of the compound into the body of a subject being treated, while lipid solutions and suspensions, as well as solid dosage forms, will result in less rapid absorption of the compound.

In one embodiment of this disclosure, a Syk kinase inhibitor can be modified with an anionic substituent that renders the inhibitor a substrate of band 3, the anion transporter of the red cell membrane. Because band 3 is dramatically more highly expressed in erythrocytes than any other cell type in the body (most cells express no band 3 whereas erythrocytes express 1,200,000 copies/cell), an otherwise poorly cell permeable Syk kinase inhibitor that can enter erythrocytes via band 3 will constitute an erythrocyte-specific Syk kinase inhibitor. Such an erythrocyte-selective Syk kinase inhibitor should exhibit reduced toxicity to non-erythroid cells requiring Syk kinase activity for normal biologic function (e. g. B cells, platelets, etc.).

### VI. Compositions Comprising Syk Kinase Inhibitors

A wide variety of compositions and administration methods may be used in conjunction with Syk kinase inhibitors of the disclosure. Such compositions may include, in addition to the Syk kinase inhibitors of the disclosure, conventional pharmaceutical excipients, and other conventional, pharmaceutically inactive agents. Additionally, the compositions may include active agents in addition to the Syk kinase inhibitors of the disclosure. These additional active agents may include additional compounds according to the disclosure, and/or one or more other pharmaceutically active agents.

The pharmaceutical compositions can be included in a container, pack, or dispenser together with instructions for administration.

The Syk kinase inhibitors of the disclosure may be administered for the purpose of preventing disease progression.

The preparation of pharmaceutical compositions that contain an active component is well understood in the art, for example, by mixing, granulating, or tablet-forming processes. The active therapeutic ingredient is often mixed with excipients that are pharmaceutically acceptable and compatible with the active ingredient. For oral administration, the active agents are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions and the like as detailed above.

The amount of the compound administered to the patient is less than an amount that would cause unmanageable toxicity in the patient. In the certain embodiments, the amount of the compound that is administered to the patient is less than the amount that causes a concentration of the compound in the patient's plasma to equal or exceed the toxic level of the compound. In one embodiment, the concentration of the compound in the patient's plasma is maintained at about 10 nM. In another embodiment, the concentration of the compound in the patient's plasma is maintained at about 25 nM. In another embodiment, the concentration of the compound in the patient's plasma is maintained at about 50 nM. In another embodiment, the concentration of the compound in the patient's plasma is maintained at about 100 nM. In another embodiment, the concentration of the compound in the patient's plasma is maintained at about 500 nM. In another embodiment, the concentration of the compound in the patient's plasma is maintained at about 1000 nM. In another embodiment, the concentration of the compound in the patient's plasma is maintained at about 2500 nM. In another embodiment, the concentration of the compound in the patient's plasma is maintained at about 5000 nM. The optimal amount of the compound that should be administered to the patient in the practice of the disclosure will depend on the particular compound used and the type of malaria being treated.

In one embodiment, the concentration of the compound in the patient's plasma is maintained at about 1-3 μM, 3-5 μM, 5-8 μM, 8-10 μM, 10-20 μM, 20-50 μM, or 50-200 μM.

In another embodiment, the peak plasma concentration of Gleevec® in the patient's plasma is maintained at about 1-3 μM, 3-5 μM, 5-8 μM, 8-10 μM, 10-20 μM, 20-50 μM, or 50-200 μM.

The compositions may be in gaseous, liquid, semi-liquid or solid form, formulated in a manner suitable for the route of administration to be used. For oral administration, capsules and tablets are typically used. For parenteral administration, reconstitution of a lyophilized powder, prepared as described herein, is typically used.

Compositions comprising Syk kinase inhibitors of the disclosure may be administered or co-administered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds and/or compositions according to the disclosure may also be administered or coadministered in slow release dosage forms. The Syk kinase inhibitors of the disclosure may be administered intravenously on the first day of treatment, with oral administration on the second day and all consecutive days thereafter.

The Syk kinase inhibitors and compositions comprising them may be administered or co-administered in any conventional dosage form. Co-administration in the context of this disclosure is intended to mean the administration of more than one therapeutic agent, one of which includes a small molecule, in the course of a coordinated treatment to achieve an improved clinical outcome. Such co-administration may also be coextensive, that is, occurring during overlapping periods of time.

Solutions or suspensions used for parenteral, intradermal, subcutaneous, or topical application may optionally include one or more of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; agents for the adjustment of tonicity such as sodium chloride or dextrose, and agents for adjusting the acidity or alkalinity of the composition, such as alkaline or acidifying agents or buffers like carbonates, bicarbonates, phosphates, hydrochloric acid, and organic acids like acetic and citric acid. Parenteral preparations may optionally be enclosed in ampules, disposable syringes or single or multiple dose vials made of glass, plastic or other suitable material.

When compounds according to the disclosure exhibit insufficient solubility, methods for solubilizing the compounds may be used. Such methods are known to those of skill in this art, and include, but are not limited to, using cosolvents, such as dimethylsulfoxide (DMSO), using surfactants, such as TWEEN, or dissolution in aqueous sodium bicarbonate. Derivatives of the compounds, such as prodrugs of the compounds may also be used in formulating effective pharmaceutical compositions.

Upon mixing or adding Syk kinase inhibitors of the disclosure to a composition, a solution, suspension, emulsion or the like may be formed. The form of the resulting composition will depend upon a number of factors, including the intended mode of administration, and the solubility of the compound in the selected carrier or vehicle. The effective concentration needed to ameliorate the disease being treated may be empirically determined

Compositions are optionally provided for administration to humans and animals in unit dosage forms, such as tablets, capsules, pills, powders, dry powders for inhalers, granules, solutions, sterile parenteral solutions or suspensions, and oral solutions or suspensions, syrup, aerosol, suspensions, and oil-water emulsions containing suitable quantities of the compounds, particularly the pharmaceutically acceptable salts, preferably the sodium salts, thereof. The pharmaceutically therapeutically active compounds and derivatives thereof are typically formulated and administered in unit-dosage forms or multiple-dosage forms. Unit-dose forms, as used herein, refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes individually packaged tablet or capsule. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pint or gallons. Hence, multiple dose form is a multiple of unit-doses that are not segregated in packaging

In addition to one or more Syk kinase inhibitors of the disclosure, the composition may comprise: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polyvinylpyrrolidine, celluloses and derivatives thereof, povidone, crospovidones and other such binders known to those of skill in the art. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to form a solution or suspension. If desired, the pharmaceutical composition to be administered may also contain minor amounts of auxiliary substances such as wetting agents, emulsifying agents, or solubilizing agents, pH buffering agents and the like, for example, acetate, sodium citrate, cyclodextrine derivatives, sorbitan monolaurate, triethanolamine sodium acetate, triethanolamine oleate, and other such agents. Actual methods of preparing such dosage forms are known in the art, or will be apparent, to those skilled in this art; for example, see Remington's Pharmaceutical Sciences, Mack Publishing Company, Easton, Pa., 15th Edition, 1975. The composition or formulation to be administered will, in any event, contain a sufficient quantity of an inhibitor of the disclosure to reduce HDAC activity in vivo, thereby treating the disease state of the subject.

Dosage forms or compositions may optionally comprise one or more Syk kinase inhibitors of the disclosure in the range of 0.005% to 100% (weight/weight) with the balance comprising additional substances such as those described herein.

In one embodiment, the amount of one or more Syk kinase inhibitors disclosed herein in a pharmaceutical composition is selected from the group consisting of: less than 80% by weight, less than 70% by weight, less than 60% by weight, less than 50% by weight, less than 40% by weight, less than 30% by weight, less than 20% by weight, and less than 10% by weight.

For oral administration, a pharmaceutically acceptable composition may optionally comprise any one or more commonly employed excipients, such as, for example pharmaceutical grades of mannitol, lactose, starch, magnesium stearate, talcum, cellulose derivatives, sodium crosscarmellose, glucose, sucrose, magnesium carbonate, sodium saccharin, talcum. Such compositions include solutions, suspensions, tablets, capsules, powders, dry powders for inhalers and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparing these formulations are known to those skilled in the art. The compositions may optionally contain 0.01%-100% (weight/weight) of one or more Syk kinase inhibitors, optionally 0.1-95%, and optionally 1-95%.

Salts, preferably sodium salts, of the inhibitors may be prepared with carriers that protect the compound against rapid elimination from the body, such as time release formulations or coatings. The formulations may further include other active compounds to obtain desired combinations of properties.

A. Formulations for Oral Administration

Oral pharmaceutical dosage forms may be as a solid, gel or liquid. Examples of solid dosage forms include, but are not limited to tablets, capsules, granules, and bulk powders. More specific examples of oral tablets include compressed, chewable lozenges and tablets that may be enteric-coated, sugar-coated or film-coated. Examples of capsules include hard or soft gelatin capsules. Granules and powders may be provided in non-effervescent or effervescent forms. Each may be combined with other ingredients known to those skilled in the art.

In certain embodiments, compounds according to the disclosure are provided as solid dosage forms, preferably capsules or tablets. The tablets, pills, capsules, troches and the like may optionally contain one or more of the following ingredients, or compounds of a similar nature: a binder; a diluent; a disintegrating agent; a lubricant; a glidant; a sweetening agent; and a flavoring agent.

Examples of binders that may be used include, but are not limited to, microcrystalline cellulose, gum tragacanth, glucose solution, acacia mucilage, gelatin solution, sucrose, and starch paste.

Examples of lubricants that may be used include, but are not limited to, talc, starch, magnesium or calcium stearate, lycopodium and stearic acid.

Examples of diluents that may be used include, but are not limited to, lactose, sucrose, starch, kaolin, salt, mannitol, and dicalcium phosphate.

Examples of glidants that may be used include, but are not limited to, colloidal silicon dioxide.

Examples of disintegrating agents that may be used include, but are not limited to, crosscarmellose sodium, sodium starch glycolate, alginic acid, corn starch, potato starch, bentonite, methylcellulose, agar and carboxymethylcellulose.

Examples of coloring agents that may be used include, but are not limited to, any of the approved certified water-soluble FD and C dyes, mixtures thereof, and water insoluble FD and C dyes suspended on alumina hydrate.

Examples of sweetening agents that may be used include, but are not limited to, sucrose, lactose, mannitol and artificial sweetening agents such as sodium cyclamate and saccharin, and any number of spray-dried flavors.

Examples of flavoring agents that may be used include, but are not limited to, natural flavors extracted from plants such as fruits and synthetic blends of compounds that produce a pleasant sensation, such as, but not limited to peppermint and methyl salicylate.

Examples of wetting agents that may be used include, but are not limited to, propylene glycol monostearate, sorbitan monooleate, diethylene glycol monolaurate, and polyoxyethylene lauryl ether.

Examples of anti-emetic coatings that may be used include, but are not limited to, fatty acids, fats, waxes, shellac, ammoniated shellac and cellulose acetate phthalates.

Examples of film coatings that may be used include, but are not limited to, hydroxyethylcellulose, sodium carboxymethylcellulose, polyethylene glycol 4000 and cellulose acetate phthalate.

If oral administration is desired, the salt of the compound may optionally be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it may optionally additionally comprise a liquid carrier such as a fatty oil. In addition, dosage unit forms may optionally additionally comprise various other materials that modify the physical form of the dosage unit, for example, coatings of sugar and other enteric agents.

Syk kinase inhibitors according to the disclosure may also be administered as a component of an elixir, suspension, syrup, wafer, sprinkle, chewing gum or the like. A syrup may optionally comprise, in addition to the active compounds, sucrose as a sweetening agent and certain preservatives, dyes and colorings and flavors.

The Syk kinase inhibitors of the disclosure may also be mixed with other active materials that do not impair the desired action, or with materials that supplement the desired action, such as other anti-malarials, including but not limited to artimisinin, chloroquine, quinine, indolone N-oxides, etc. Syk kinase inhibitors may also be co-administered with pharmaceuticals designed to suppress the toxic effects of Syk kinase inhibitors on other nonerythroid cells. For example, if a compound is used for treating asthma or hypertension, it may be used with other bronchodilators and antihypertensive agents, respectively.

Examples of pharmaceutically acceptable carriers that may be included in tablets comprising compounds of the present disclosure include, but are not limited to binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets may be compressed tablets to which different layers of pharmaceutically acceptable substances are applied. Film-coated tablets may be compressed tablets that have been coated with polymers or other suitable coating. Multiple compressed tablets may be compressed tablets made by more than one compression cycle utilizing the pharmaceutically acceptable substances previously mentioned. Coloring agents may also be used in tablets. Flavoring and sweetening agents may be used in tablets, and are especially useful in the formation of chewable tablets and lozenges.

Examples of liquid oral dosage forms that may be used include, but are not limited to, aqueous solutions, emulsions, suspensions, solutions and/or suspensions reconstituted from non-effervescent granules and effervescent preparations reconstituted from effervescent granules.

Examples of aqueous solutions that may be used include, but are not limited to, elixirs and syrups. As used herein, elixirs refer to clear, sweetened, hydroalcoholic preparations. Examples of pharmaceutically acceptable carriers that may be used in elixirs include, but are not limited to solvents. Particular examples of solvents that may be used include glycerin, sorbitol, ethyl alcohol and syrup. As used herein, syrups refer to concentrated aqueous solutions of a sugar, for example, sucrose. Syrups may optionally further comprise a preservative.

Emulsions refer to two-phase systems in which one liquid is dispersed in the form of small globules throughout another liquid. Emulsions may optionally be oil-in-water or water-in-oil emulsions. Examples of pharmaceutically acceptable carriers that may be used in emulsions include, but are not limited to non-aqueous liquids, emulsifying agents and preservatives.

Examples of pharmaceutically acceptable substances that may be used in non-effervescent granules, to be reconstituted into a liquid oral dosage form, include diluents, sweeteners and wetting agents.

Examples of pharmaceutically acceptable substances that may be used in effervescent granules, to be reconstituted into a liquid oral dosage form, include organic acids and a source of carbon dioxide.

Coloring and flavoring agents may optionally be used in all of the above dosage forms.

Particular examples of preservatives that may be used include glycerin, methyl and propylparaben, benzoic add, sodium benzoate and alcohol.

Particular examples of non-aqueous liquids that may be used in emulsions include mineral oil and cottonseed oil.

Particular examples of emulsifying agents that may be used include gelatin, acacia, tragacanth, bentonite, and surfactants such as polyoxyethylene sorbitan monooleate.

Particular examples of suspending agents that may be used include sodium carboxymethylcellulose, pectin, tragacanth, Veegum and acacia. Diluents include lactose and sucrose. Sweetening agents include sucrose, syrups, glycerin and artificial sweetening agents such as sodium cyclamate and saccharin.

Particular examples of wetting agents that may be used include propylene glycol monostearate, sorbitan monooleate, diethylene glycol monolaurate, and polyoxyethylene lauryl ether.

Particular examples of organic acids that may be used include citric and tartaric acid.

Sources of carbon dioxide that may be used in effervescent compositions include sodium bicarbonate and sodium carbonate. Coloring agents include any of the approved certified water soluble FD and C dyes, and mixtures thereof.

Particular examples of flavoring agents that may be used include natural flavors extracted from plants such fruits, and synthetic blends of compounds that produce a pleasant taste sensation.

For a solid dosage form, the solution or suspension, in for example propylene carbonate, vegetable oils or triglycerides, is preferably encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. Re 28,819 and 4,358,603.

B. Injectables. Solutions, and Emulsions

The disclosure also is directed to compositions designed to administer the Syk kinase inhibitors by parenteral administration, generally characterized by subcutaneous, intramuscular or intravenous injection. Injectables may be prepared in any conventional form, for example as liquid solutions or suspensions, solid forms suitable for solution or suspension in liquid prior to injection, or as emulsions.

Examples of excipients that may be used in conjunction with injectables according to the present disclosure include, but are not limited to water, saline, dextrose, glycerol or ethanol. The injectable compositions may also optionally comprise minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) is also contemplated herein. The percentage of active compound contained in such parenteral compositions is highly dependent on the specific nature thereof, as well as the activity of the compound and the needs of the subject.

Parenteral administration of the formulations includes intravenous, subcutaneous and intramuscular administrations. Preparations for parenteral administration include sterile solutions ready for injection, sterile dry soluble products, such as the lyophilized powders described herein, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

When administered intravenously, examples of suitable carriers include, but are not limited to physiological saline or phosphate buffered saline (PBS), and solutions containing thickening and solubilizing agents, such as glucose, polyethylene glycol, and polypropylene glycol and mixtures thereof.

Examples of pharmaceutically acceptable carriers that may optionally be used in parenteral preparations include, but are not limited to aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles that may optionally be used include Sodium Chloride Injection, Ringers Injection, Isotonic Dextrose Injection, Sterile Water Injection, Dextrose and Lactated Ringers Injection.

Examples of nonaqueous parenteral vehicles that may optionally be used include fixed oils of vegetable origin, cottonseed oil, corn oil, sesame oil and peanut oil.

Antimicrobial agents in bacteriostatic or fungistatic concentrations may be added to parenteral preparations, particularly when the preparations are packaged in multiple-dose containers and thus designed to be stored and multiple aliquots to be removed. Examples of antimicrobial agents that may be used include phenols or cresols, mercurials, benzyl alcohol, chlorobutanol, methyl and propyl p-hydroxybenzoic acid esters, thimerosal, benzalkonium chloride and benzethonium chloride.

Examples of isotonic agents that may be used include sodium chloride and dextrose. Examples of buffers that may be used include phosphate and citrate. Examples of antioxidants that may be used include sodium bisulfate. Examples of local anesthetics that may be used include procaine hydrochloride. Examples of suspending and dispersing agents that may be used include sodium carboxymethylcellulose, hydroxypropyl methylcellulose and polyvinylpyrrolidone. Examples of emulsifying agents that may be used include Polysorbate 80 (TWEEN 80). A sequestering or chelating agent of metal ions includes EDTA.

Pharmaceutical carriers may also optionally include ethyl alcohol, polyethylene glycol and propylene glycol for water miscible vehicles and sodium hydroxide, hydrochloric acid, citric acid or lactic acid for pH adjustment.

The concentration of an inhibitor in the parenteral formulation may be adjusted so that an injection administers a pharmaceutically effective amount sufficient to produce the desired pharmacological effect. The exact concentration of an inhibitor and/or dosage to be used will ultimately depend on the age, weight and condition of the patient or animal as is known in the art.

Unit-dose parenteral preparations may be packaged in an ampoule, a vial or a syringe with a needle. All preparations for parenteral administration should be sterile, as is known and practiced in the art.

Injectables may be designed for local and systemic administration. Typically a therapeutically effective dosage is formulated to contain a concentration of at least about 0.1% w/w up to about 90% w/w or more, preferably more than 1% w/w of the small molecule to the treated tissue(s). The small molecule may be administered at once, or may be divided into a number of smaller doses to be administered at intervals of time. It is understood that the precise dosage and duration of treatment will be a function of the location of where the composition is parenterally administered, the carrier and other variables that may be determined empirically using known testing protocols or by extrapolation from in vivo or in vitro test data. It is to be noted that concentrations and dosage values may also vary with the age of the individual treated. It is to be further understood that for any particular subject, specific dosage regimens may need to be adjusted over time according to the individual need and the professional judgment of the person administering or supervising the administration of the formulations. Hence, the concentration ranges set forth herein are intended to be exemplary and are not intended to limit the scope or practice of the claimed formulations.

The small molecule may optionally be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the disease state and may be empirically determined.

C. Lyophilized Powders

The Syk kinase inhibitors of the disclosure may also be prepared as lyophilized powders, which can be reconstituted for administration as solutions, emulsions and other mixtures. The lyophilized powders may also be formulated as solids or gels.

Sterile, lyophilized powder may be prepared by dissolving the compound in a sodium phosphate buffer solution containing dextrose or other suitable excipient. Subsequent sterile filtration of the solution followed by lyophilization under standard conditions known to those of skill in the art provides the desired formulation. Briefly, the lyophilized powder may optionally be prepared by dissolving dextrose, sorbitol, fructose, corn syrup, xylitol, glycerin, glucose, sucrose or other suitable agent, about 1-20%, preferably about 5 to 15%, in a suitable buffer, such as citrate, sodium or potassium phosphate or other such buffer known to those of skill in the art at, typically, about neutral pH. Then, a small molecule is added to the resulting mixture, preferably above room temperature, more preferably at about 30-35 C., and stirred until it dissolves. The resulting mixture is diluted by adding more buffer to a desired concentration. The resulting mixture is sterile filtered or treated to remove particulates and to insure sterility, and apportioned into vials for lyophilization. Each vial may contain a single dosage or multiple dosages of the inhibitor.

D. Topical Administration

The Syk kinase inhibitors of the present disclosure may also be administered as topical mixtures. Topical mixtures may be used for local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The Syk kinase inhibitors may be formulated as aerosols for topical application, such as by inhalation (see, U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will typically have diameters of less than 50 microns, preferably less than 10 microns.

The Syk kinase inhibitors may also be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the small molecule alone or in combination with other pharmaceutically acceptable excipients can also be administered.

E. Formulations for Other Routes of Administration

Depending upon the disease state being treated, other routes of administration, such as topical application, transdermal patches, and rectal administration, may also be used. For example, pharmaceutical dosage forms for rectal administration are rectal suppositories, capsules and tablets for systemic effect. Rectal suppositories are used herein mean solid bodies for insertion into the rectum that melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax, (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm. Tablets and capsules for rectal administration may be manufactured using the same pharmaceutically acceptable substance and by the same methods as for formulations for oral administration.

### V. Combination Therapy

The Syk kinase inhibitors of the disclosure can be administered alone or in combination with other therapies suitable for the disease or disorder being treated. Where separate dosage formulations are used, the Syk kinase inhibitors and the other therapeutic agent can be administered at essentially the same time (concurrently) or at separately staggered times (sequentially). The pharmaceutical combination is understood to include all these regimens. Administration in these various ways are suitable for the present disclosure as long as the beneficial therapeutic effect of the small molecule and the other therapeutic agent are realized by the patient at substantially the same time. In an embodiment, such beneficial effect is achieved when the target blood level concentrations of each active drug are maintained at substantially the same time.

The Syk kinase inhibitors of the disclosure are also useful in combination with known therapeutic agents and anti-malaria agents (e.g, antimalarial drugs). Combinations of the presently disclosed Syk kinase inhibitors with other anti-malaria agents are within the scope of the disclosure. A person of ordinary skill in the art would be able to discern the combinations of agents that would be useful based on the particular characteristics of the drugs and the disease.

In one embodiment, a Syk kinase inhibitor can be used with one or more antimalaria agents. In another embodiment, a Syk kinase inhibitor can be used with two or more anti-malaria agents.

In one embodiment, an anti-malaria agent can be administered prior to administration of the Syk kinase inhibitor. An anti-malaria agent can be administered 24, 48, 72 or 96 hours prior to administration of the Syk kinase inhibitor.

In another embodiment, an anti-malaria agent can be administered from 3 to 5 days, from 5 to 7 days, from 7-14 days, from 14-21 days, or from 21-28 days prior to administration of the Syk kinase inhibitor.

In yet another embodiment, an anti-malaria agent can be administered from 1 to 2 weeks, from 2-4 weeks, from 4-6 weeks, or from 6-8 weeks prior to administration of the Syk kinase inhibitor.

In still another embodiment, an anti-malaria agent can be administered from 1 to 2 months, from 2 to 4 months, from 4 to 6 months or from 6 to 12 months prior to administration of the Syk kinase inhibitor.

Such anti-malaria agents include but are not limited to quinine, quinimax (quinine, quinidine, cinchoine and cinchonidine), quinidine (direct derivative of quinine), alkaloids, Warburg's Tincture (quinine as key ingredient), chloroquine, chloroquine phosphate, nivaquine, Chloroquine FNA, Resochin, Dawaquin, 4-aminoquinolone compounds, Amodiaquine, Pyrimethamine, sulfadoxine, Proguanil (chloroguanide), proguanil hydrochloride, Paludrine, biguanide, synthetic derivatives of pyrimidine, sulfonamide, sulfadoxine, sulfamethoxypyridazine, mefloquine, combination of mefloquine and artesunate, chloroquine/proguanil or sulfa drug-pyrimethamine combinations, atovaquone, atovaquone and proguanil, atovaquone-proguanil (Malarone), artemetherlumefantrine (Coartem®), mefloquine (Lariam), Primaquine, 8-aminoquinolone, Artemisinin and derivatives, qinghaosu, Artemether, methyl ether derivative of dihydroartemesinin, Artesunate, hemisuccinate derivative of the active metabolite dihydroartemisin, Dihydroartemisinin, Arteether, ethyl ether derivative of dihydroartemisinin, Halofantrine, phenanthrene methanol, Doxycycline, tetracycline compound derived from oxytetracycline, Clindamycin, derivative of lincomycin, and indolone N-oxides (INODS) of various structures, etc.

Further, the Syk kinase inhibitors disclosed herein can be used in combination with any agent that acts as an inhibitor of dihydrofolate reductase, DNA replication, cell division, and enzyme dihyropteroate.

### VI. Dosages and Dosing Schedules

The dosage regimen utilizing Syk kinase inhibitors of the disclosure can be selected in accordance with a variety of factors including type, species, age, weight, sex and the type of malaria being treated; the severity (i.e., stage) of the disease to be treated; the route of administration; the renal and hepatic function of the patient; and the particular compound or salt thereof employed. An ordinarily skilled physician or veterinarian can readily determine and prescribe the effective amount of the drug required to treat, for example, to prevent, inhibit (fully or partially) or arrest the progress of the disease.

For oral administration, suitable daily dosages are for example between about 2-4000 mg administered orally once-daily, twice-daily or three times-daily, continuous (every day) or intermittently (e.g. 3-5 days a week). The small molecule or pharmaceutical compositions comprising a small molecule is administered once daily (QD), or divided into multiple daily doses such as twice daily (BID), and three times daily (TID). For administration once a day, a suitably prepared medicament would therefore contain all of the needed daily dose. For administration twice a day, a suitably prepared medicament would therefore contain half of the needed daily dose. For administration three times a day, a suitably prepared medicament would therefore contain one third of the needed daily dose.

In addition, the administration can be continuous, i.e., every day, or intermittently. The terms “intermittent” or “intermittently” as used herein means stopping and stirring at either regular or irregular intervals. For example, intermittent administration of a small molecule may be administration one to six days per week or it may mean administration in cycles (e.g., daily administration for two to eight consecutive weeks, then a rest period with no administration for up to one week) or it may mean administration on alternate days.

The compounds can also be administered in intranasal form via topical use of suitable intranasal vehicles, or via transdermal routes, using those forms of transdermal skin patches well known to those of ordinary skill in that art. To be administered in the form of a transdermal delivery system, the dosage administration will, or course, be continuous rather than intermittent throughout the dosage regime.

It should be apparent to a person skilled in the art that the various modes of administration, dosages and dosing schedules described herein merely set forth specific embodiments and should not be construed as limiting the broad scope of the disclosure. Any permutations, variations and combinations of the dosages and dosing schedules are included within the scope of the disclosure.

The disclosure is now described with reference to the following Examples. These Examples are provided for the purpose of illustration only and the disclosure should in no way be construed as being limited to these Examples, but rather should be construed to encompass any and all variations that become evident as a result of the teaching provided herein. All references including but not limited to U.S. patents, allowed U.S. patent applications, or published U.S. patent applications are incorporated within this specification by reference in their entirety.

### EXAMPLES

The following examples are illustrative only and are not intended to limit the scope of the disclosure as defined by the claims.

### Example 1

In this example, the following questions were addressed: (i) whether parasitized RBCs at the peak of their tyrosine phosphorylation have weakened erythrocyte membranes; (ii) if so, do they rely on this membrane weakening to complete their life cycle, and (iii) can inhibiting Syk kinase prevent the parasite from completing its life cycle?Materials and Methods

### Inhibition of Syk in RBCs.

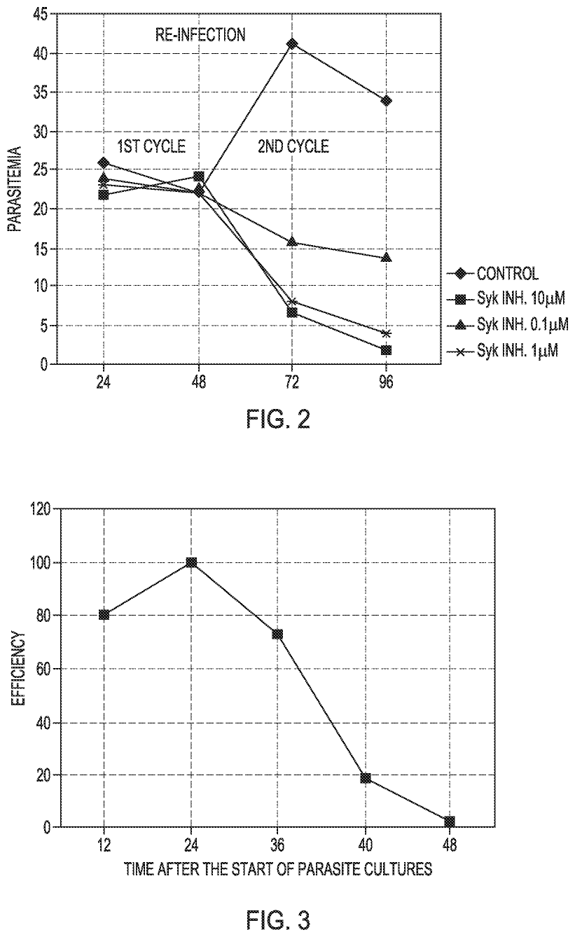

Infected and non-infected RBCs were treated with Syk kinase inhibitor II (FIG. 2) at different concentrations and at different times after the start of the cultures

Cultivation of P. falciparum-Infected RBCs.

Freshly drawn blood (Rh+) from healthy adults of both sexes was used following informed consent in all studies. To prevent coagulation, blood was treated with heparin and stored for 1-6 hours in citrate-phosphate-dextrose with adenine (CPDA-1) prior to its use. RBCs were separated from plasma and leukocytes by washing three times in wash medium (RPMI 1640 medium containing 2 mM glutamine, 24 mM NaHCO₃, 25 mM HEPES, 20 mM glucose, and 32 mg/mL gentamicin, pH 6.80). P. falciparum strain Palo Alto (mycoplasma free) was cultured at a hematocrit of 0.5%. Synchronous cultures were started by injecting density separated schizonts at a parasitemia ranging from 20 to 25%.

To assess total parasitemia and the relative numbers of ring and trophozoite stage cells, slides were prepared from cultures at the indicated times and stained with Diff-Quik reagent prior to analysis. 1000 cells were then scored by microscopic analysis of the cell staining patterns.

For membrane studies or measurement of microvesicle release, infected cells were separated by density gradient on Percoll. Standard hypotonic membranes were prepared at 4° C. on ice as follows: 150 μL of packed RBCs were diluted into 1.5 mL of cold hemolysis buffer (5 mmol/L sodium phosphate, 1 mmol/L EDTA, pH 8.0) containing a cocktail of protease and phosphatase inhibitors (Sigma-Aldrich, St. Louis, Mo.), and then washed up to 4 more times in the same buffer using a refrigerated Eppendorf microfuge at 25,000×g for pelleting of the cells. The preparations were stored frozen at −80° C. until use. Membrane protein content was quantified using the DC Protein Assay (Biorad).

Measurement of Merozoites Egress:

Schizonts were isolated from P. falciparum cultures (strain Palo Alto) by density gradient and then cultivated for 8 hours. At 2 hour time intervals, the number of free merozoites was counted by flow cytometric analysis using a FACSCalibur cytometer (BD Biosciences) and the Cell Quest analysis software (BD Biosciences). Merozoite DNA was stained with propidium iodide

Micro-Vesicle Quantification.

Infected and non-infected RBCs were washed 3× in RPMI 1640-HEPES medium and then resuspended at 10% hematocrit in the same medium containing a protease inhibitor cocktail (Sigma-Aldrich). Packed cells were re-suspended at a 30% hematocrit in 2 mM PBS-G) and incubated for 2.5 hours at 37° C. while shaking. Cells were then centrifuged at 2000×g to pellet the RBCs followed by a centrifugation step at 20,000×g for 10 minutes to pellet the RBC membranes. RBC vesicles present in the supernatant were labeled with anti-glycophorin A antibodies or with eosine-maleimide for labeling band 3, and quantified using a FACSCalibur cytometer (BD Biosciences) and the Cell Quest analysis software (BD Biosciences). Vesicles were selected using forward and side light scatter (FSC and SSC signals set to logarithmic amplification). A total of ˜40,000 events were analyzed.

Immunoblot Analysis.

Proteins were separated using either 1-D or 2-D gel electrophoresis and then transferred onto nitrocellulose membranes. The nitrocellulose membranes were then probed using either fluorescein-5′-maleimide (0.25 mg/ml)(Pierce) or anti-phosphotyrosine antibody (Santa Cruz, Calif.) and anti-Syk (Santa Cruz, Calif.) antibodies and with anti-band 3 (Sigma Aldrich). The blots were then analyzed using an 800 nm laser scanner (Odyssey, Licor, USA). To ascertain the specificity of anti-phosphotyrosine staining, proteins were de-phosphorylated prior to gel electrophoresis by incubating the samples for 20 min at 30° C. with 6 μL (400 units) lambda phosphatase (in 50 mM Tris buffer, pH 7.5, 0.1 mM Na₂EDTA, 5 mM dithiothreitol, and 2 mM MnCl₂).

Results

To evaluate the potential of Syk kinase inhibitors as a therapy for treating malaria, the effect of Syk kinase inhibitors on preventing re-infection of additional erythrocytes was evaluated. Based on the hypothesis that destabilization of the RBCM is beneficial to the parasite during egress, blocking RBCM destabilization by inhibiting Syk-mediated tyrosine phosphorylation of band 3 may result in a reduction in the spread of infected cells. To test this, erythrocyte cultures were infected with P. falciparum and then treated 24 hours later with the Syk kinase inhibitor (0, 0.1, 1, and 10 uM concentrations) (FIG. 2).

In this set of experiments Syk kinase inhibitors have been added 24 hours after the start of the parasite cultures. Data are means of 2 or 4 experiments with standard deviations.

It can be observed that during the first cycle (first 48 hours), Syk kinase inhibitors do not cause changes of parasitemia. Nevertheless, treated infected-RBCs show a delay of development. After re-infection at the second cycle, the effect of Syk kinase inhibitors on parasitemia is pronounced (inhibition of the re-infection rate). The large variations at lower Syk kinase inhibitors concentrations (0.1 μM) may be also due to insufficient optimization of our experimental conditions.

Not to be bound by any particular theory, it is believed that Syk kinase inhibitors work mainly at the egress phase because if a new culture is started using normal merozoites to infect RBC that have been treated with Syk kinase inhibitors, no inhibition is seen.

The data presented represents the mean of two to four experiments. The level of parasitemia was then calculated at 24, 48, 72 and 96 hours after addition of the Syk kinase inhibitor.

During the first parasite cycle (first 48 hours), the Syk kinase inhibitor had no effect on the level of parasitemia (inhibition of the re-infection rate). However during the second cycle when re-infection occurred in the negative control cells, the Syk kinase inhibitor significantly dropped the level of parasitemia at all concentrations examined. Indeed, at the 1 μM and 10 μM concentrations, the parasitemia level dropped to ˜80% of the initial level 72 hours after treatment. Upon microscopic examination of the treated cells, it's apparent that the merozoite particles remain entrapped within the host erythrocyte, confirming that parasite egress has been compromised. Further, if a new culture is treated with Syk kinase inhibitors prior to infection with normal merozoites, there is no effect in blocking the parasitemia. Combined this evidence suggests that the Syk kinase inhibitors are primarily blocking the egress phase of the parasite cycle.

The efficiency of the Syk kinase inhibitor at different stages of the parasite development cycle was examined. Specifically, the Syk kinase inhibitor (1 μM concentration) was added at different times following the start of the parasite cultures (12, 24, 36, 40 and 48 hours (FIG. 3). Growth inhibition was calculated as a % re-invasion rate relative to untreated cultures. As expected, treatment of the parasite cultures at 40 and 48 hours post initial infection had little impact on inhibiting parasite re-infection. However, when infected cultures are treated at 24 hours after the initial infection, nearly 100% of parasite re-infection is inhibited. This time point was confirmed in a flow cytometric analysis where the number of merozoites released from isolated schizonts, were measured with or without Syk kinase inhibitor treatment. This time course analysis suggests that there is a window of effectiveness for the Syk kinase inhibitor. The Syk kinase inhibitor appears to be most effective just prior to the egress phase and may be active in preparing the RBCM for egress. This is consistent with our proposed mechanism of the Syk kinase inhibitor blocking the necessary destabilization of the erythrocyte cell membrane required for egress.

In a separate set of experiments, the number of merozoites that are released from isolated schizonts with or without inhibitors was measured by FACS (data not shown). Those data confirmed the above results: maximal effect of inhibitors if they are added at 24 hours.

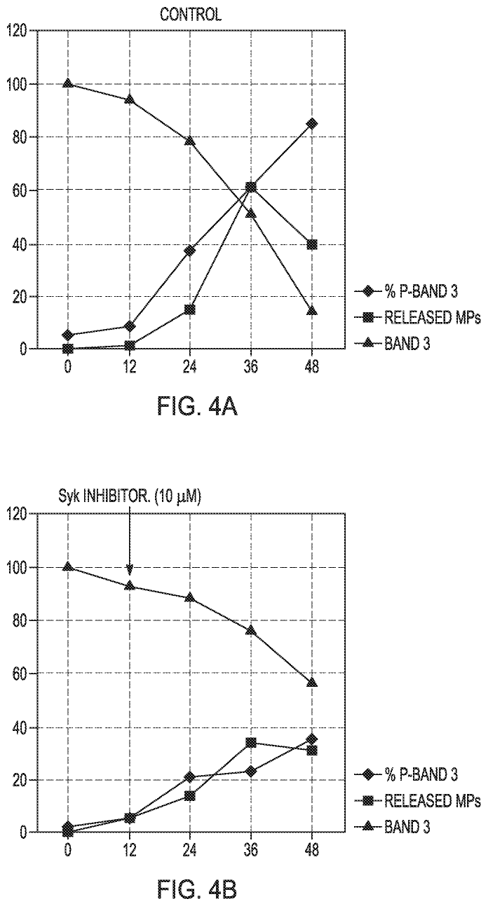

To further examine the timing and mechanism by which the Syk kinase inhibitors are blocking parasite egress and subsequent spread, membrane associated events associated with egress were examined: vesiculation, loss of band 3, and tyrosine phosphorylation of band 3 (FIG. 4).

Brief Summary of FIG. 4.

In this set of experiments, Syk kinase inhibitors were added 12 hours after the start of the parasite cultures. The content of band 3 was measured by western blotting. The percent of band 3 phosphorylation was also measured. The reference (100% of phosphorylation) was the degree of band 3 phosphorylation measured treating RBCs with vanadate 2 mM for 2 hours. Band 3 phosphorylation levels have been normalized for the band 3 content (wester blot).

The amount of MPs released by infected RBCs was also measured. Cells were sampled from the cultures, washed and incubated 2 hours under shacking. MPs were isolated from the supernatant by differential centrifugation (as in the INODs paper) and their amount was estimated measuring their band 3 content (wester blots). Data are mean of 2 experiments with SD (in the second experiment we could measure only the ast times).

Not to be bound by any particular theory, Syk kinase inhibitors cause a reduction of vesiculation and band 3 loss. Concomitantly there is a reduction of band 3 phosphorylation

Detailed Description of FIG. 4.

Erythrocytes were infected with the parasite and treated 12 hours later with 10 μM Syk kinase inhibitor. To measure vesiculation, microparticles were isolated from the culture supernatants and quantified based on their band 3 content (determined by Western blotting) [Pantaleo, Free Radic Biol Med 2012, 52: 527-536]. As compared to untreated parasite cultures, vesiculation was inhibited by 50%.

The expectation is that band 3 tyrosine phosphorylation would also be diminished in the Syk kinase inhibitor treated cultures. This is indeed the case. The % of band tyrosine phosphorylation was measured via Western blotting with an anti-phosphotyrosine antibody. As a reference representing maximal band 3 phosphorylation, cells were treated with 2 mM vanadate for 2 hours. The cultures were then normalized to the vandate induced level of phosphorylation. In untreated parasite cultures, the level of band 3 phosphorylation was increased over time to an eventual maximum level of 85%. This is consistent with the hypothesis that just prior to egress the parasite would phosphorylate band 3, disrupt its interaction with ankyrin and result in a destabilized membrane that is more susceptible to breech as required during egress.

Treatment of the cells with the Syk kinase inhibitor, significantly diminished the level of tyrosine phosphorylated band 3 reaching a maximum of ˜35% at 48 hours post infection. Combined with the re-infection data presented earlier, the Syk kinase inhibitor effectively disrupts band-3 phosphorylation, parasite egress, and subsequently parasite re-invasion.

### Example 2

Integral membrane proteins provide the cell with a vital link to its environment. Because they are responsible for essential functions such as cell polarity, signal transduction, and vectorial transport, their targeting and placement is of critical importance. It is well accepted that their interactions with other proteins, such as those making up the cytoskeleton, are largely responsible for anchorage and stabilization at specific plasma membrane domains. Two of these membrane proteins are band 3 and Ankyrin.

Band 3 is a very abundant 93-kDa integral membrane glycoprotein that mediates chloride/bicarbonate exchange in erythrocytes. It is composed of two structurally and functionally distinct domains. The carboxyl-terminal 55 kDa spans the membrane at least 12 times, and is responsible for catalyzing the rapid exchange of anions across the plasma membrane. The 40-kDa NHz-terminal domain is cytosolic and directly interacts with high affinity with ankyrin, a 215-kDa cytosolic polypeptide. The band 3 binding domain of ankyrin has been characterized recently as a sequence of 33 repeats of 22 amino acids. A separate domain of ankyrin binds the R subunit of spectrin, the main element of the erythrocyte membrane skeleton. Spectrin is found as a tetramer consisting of two α/β subunits or as higher order oligomers.

Ankyrins are a family of adaptor proteins that mediate the attachment of integral membrane proteins to the spectrin-actin based membrane cytoskeleton. Ankyrins have binding sites for the beta subunit of spectrin and at least 12 families of integral membrane proteins. This linkage is required to maintain the integrity of the plasma membranes and to anchor specific ion channels, ion exchangers and ion transporters in the plasma membrane

Ankyrins contain four functional domains: an N-terminal domain that contains 24 tandem ankyrin repeats, a central domain that binds to spectrin, a death domain that binds to proteins involved in apoptosis, and a C-terminal regulatory domain that is highly variable between different ankyrin proteins. Ankyrins are encoded by three genes (ANK1, ANK2 and ANK3) in mammals. Each gene in turn produces multiple proteins through alternative splicing.

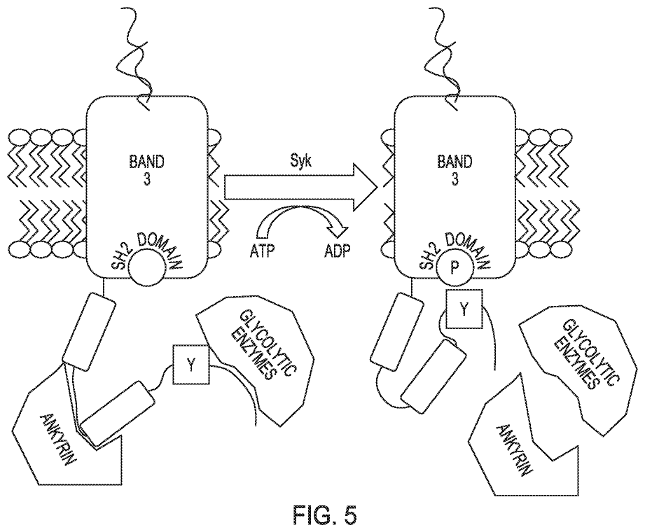

As depicted in FIG. 5, band 3 and ankyrin interact to stabilize the cell membrane. Upon tyrosine phosphorylation of band 3 cytoplasmic domain, the phosphorylated residue is able to interact with the SH2 domain of band 3. This interaction then disrupts band 3's interaction with ankyrin, breaking a critical bridge between the membrane and cytoskeleton thus destabilizing the red cell membrane. As discussed above, this disruption of the cell membrane is essential for the parasite to escape infected RBCs.

Materials and Methods

Inhibition of Syk in RBCs.

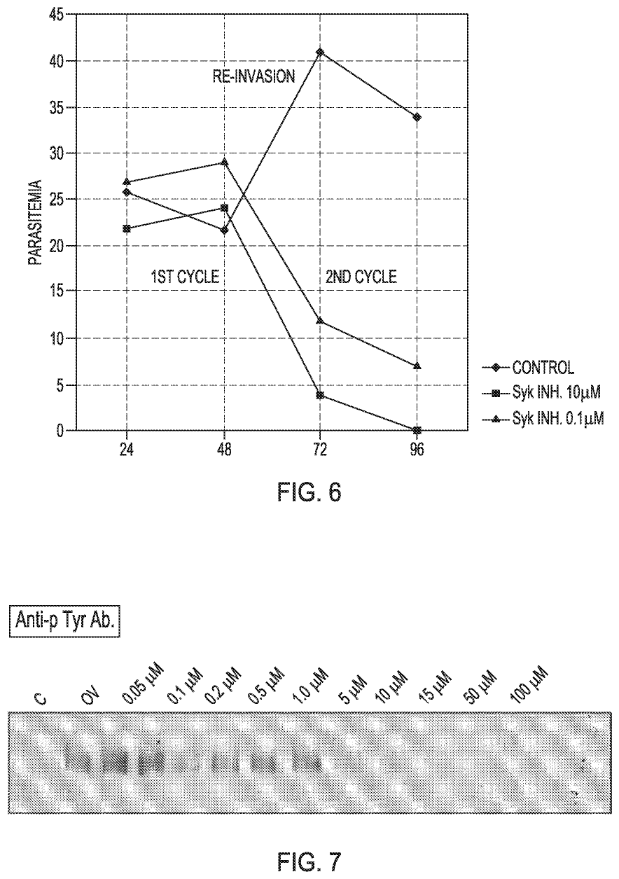

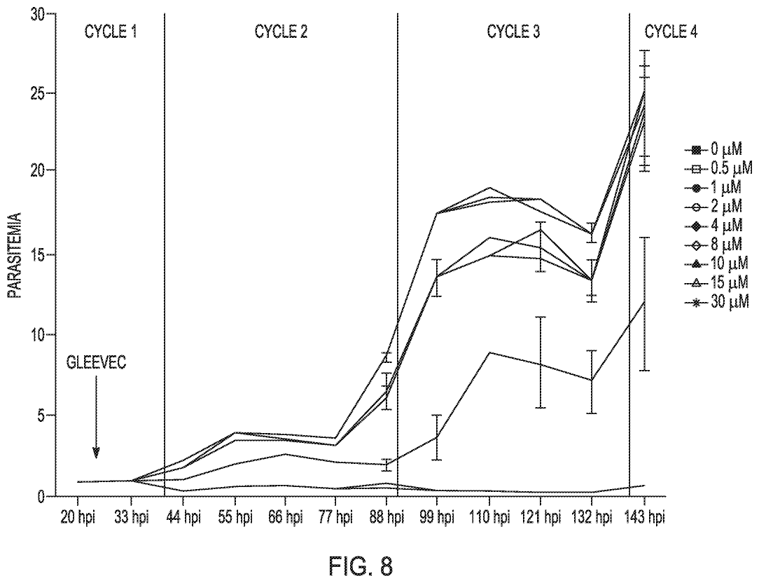

Infected and non-infected RBCs were treated with Syk kinase inhibitor IV (FIG. 6) at different concentrations and at different times after the start of the cultures.

Cultivation of P. falciparum-infected RBCs.

Freshly drawn blood (Rh+) from healthy adults of both sexes was used following informed consent in all studies. To prevent coagulation, blood was treated with heparin and stored for 1-6 hours in citrate-phosphate-dextrose with adenine (CPDA-1) prior to its use. RBCs were separated from plasma and leukocytes by washing three times in wash medium (RPMI 1640 medium containing 2 mM glutamine, 24 mM NaHCO₃, 25 mM HEPES, 20 mM glucose, and 32 mg/mL gentamicin, pH 6.80). P. falciparum strain Palo Alto (mycoplasma free) was cultured at a hematocrit of 0.5%. Synchronous cultures were started by injecting density separated schizonts at a parasitemia ranging from 20 to 25%.

To assess total parasitemia and the relative numbers of ring and trophozoite stage cells, slides were prepared from cultures at the indicated times and stained with Diff-Quik reagent prior to analysis. 1000 cells were then scored by microscopic analysis of the cell staining patterns.

For membrane studies or measurement of microvesicle release, infected cells were separated by density gradient on Percoll. Standard hypotonic membranes were prepared at 4° C. on ice as follows: 150 μL of packed RBCs were diluted into 1.5 mL of cold hemolysis buffer (5 mmol/L sodium phosphate, 1 mmol/L EDTA, pH 8.0) containing a cocktail of protease and phosphatase inhibitors (Sigma-Aldrich, St. Louis, Mo.), and then washed up to 4 more times in the same buffer using a refrigerated Eppendorf microfuge at 25,000×g for pelleting of the cells. The preparations were stored frozen at −80° C. until use. Membrane protein content was quantified using the DC Protein Assay (Biorad).

Results

To evaluate the potential of Syk kinase inhibitor IV as a therapy for treating malaria, the effect of Syk kinase inhibitor IV on preventing re-infection of additional erythrocytes was evaluated. Based on the hypothesis that destabilization of the RBCM is beneficial to the parasite during egress, blocking RBCM destabilization by inhibiting Syk-mediated tyrosine phosphorylation of band 3 may result in a reduction in the spread of infected cells.

To test this, erythrocyte cultures were infected with P. falciparum and then treated 24 hours later with the Syk kinase inhibitor (0, 0.1, and 10 uM concentrations) (FIG. 6).

In this set of experiments Syk kinase inhibitor IV was added 24 hours after the start of the parasite cultures. It can be observed that during the first cycle (first 48 hours), Syk kinase inhibitor IV did not cause changes of parasitemia. Nevertheless, treated infected-RBCs show a delay of development. After re-infection at the second cycle, the effect of Syk kinase inhibitor IV on parasitemia is pronounced (inhibition of the re-infection rate).

The data presented represents the mean of two to four experiments. The level of parasitemia was then calculated at 24, 48, 72 and 96 hours after addition of the Syk kinase inhibitor.

During the first parasite cycle (0-48 hours), the Syk kinase inhibitor had minimal effect on the level of parasitemia (inhibition of the re-infection rate). However during the second cycle when re-infection occurred in the negative control cells, the Syk kinase inhibitor significantly dropped the level of parasitemia at both concentrations examined. Indeed, at the 10 μM concentration, the parasitemia level dropped to ˜80 of the initial level 72 hours after treatment. This evidence further suggests that the Syk kinase inhibitors are primarily blocking the egress phase of the parasite cycle.

### Example 3

In this Example, the ability of imatinib mesylate (Gleevec) to inhibit phosphorylation of Band 3 was tested.

Materials and Methods

Blood was collected from healthy volunteers after informed consent and immediately processed. Briefly, blood was centrifuged at 1200×g to separate red cells from the buffy coat and plasma, and subsequently washed three times in PBS (137 mM NaCl, 2.7 mM KC, 8.1 mM K₂HPO₄, and 1.5 mM KH₂PO₄, pH 7.4) to remove any remaining white blood cells.

Packed erythrocytes were re-suspended at 30% hematocrit in PBS containing 5 mM glucose and treated with varying concentrations of Gleevec® (Santa Cruz Biotechnology). Stock solutions of Gleevec, prepared in double distilled water pH 5.5, were prepared fresh. Untreated control erythrocytes and Gleevec® treated red cells were incubated for 1 hour at 37° C.

To induce tyrosine phosphorylation, erythrocytes were then incubated with 2 mM orthovanadate and again incubated for 1 hour at 37° C. Cells were packed via centrifugation and added to 2× Sample buffer containing 5% betamercaptoethanol and PMSF protease inhibitor and stored at −20° C. or analyzed immediately. Proteins separated by SDS-PAGE gel electrophoresis were transferred to nitrocellulose membranes and probed with anti-phosphotyrosine antibody (Santa Cruz Biotechnology) diluted 1:1000 in TBST. Secondary antibody was conjugated to horse radish peroxidase enzyme, the blot incubated in chemiluminescent substrate, and proteins detected using film.

As shown in FIG. 7, band 3 tyrosine phosphorylation is inhibited upon increasing concentrations of Gleevec. Phosphorylation is completely inhibited upon incubation with 10 uM Gleevec. The control cells are designated “C,” and were untreated. Cells treated only with orthovanadate are designated “OV.”

### Example 4

Imatinib mesylate (Gleevec) has been approved for numerous clinical uses at various clinical dosages. Gleevec® is typically administered orally through a tablet, which may be administered at various times points throughout a 24 hour period. Clinical trials with Gleevec® were conducted at various dosages, and up to about 1000 mg.

Dosage and administration for Gleevec® varies depending on the clinical condition. Below is a brief overview of the dosages:
Adults with Ph+CML (Chronic Phase: CP): 400 mg/day
Adults with Ph+CML (AP or BC): 600 mg/day
Pediatrics with Ph+CML (CP): 340 mg/m2/day
Adults with Ph+ALL: 600 mg/day
Pediatrics with Ph+ALL: 340 mg/m2/day
Adults with MDS/MPD: 400 mg/day
Adults with ASM: 100 mg/day or 400 mg/day
Adults with HES/CEL: 100 mg/day or 400 mg/day
Adults with Dermatofibrosarcoma: 800 mg/day
Adults with metastatic and/or unresectable GIST: 400 mg/day
Adjuvant treatment of adults with GIST: 400 mg/day
Patients with mild to moderate hepatic impairment: 400 mg/day
Patients with severe hepatic impairment: 300 mg/day

Doses of 400 mg or 600 mg should be administered once daily, whereas a dose of 800 mg should be administered as 400 mg twice a day.

Materials and Methods

In this experiment, Gleevec® was added 20 hours after the start of parasite cultures (hpi refers to hours post infection). Briefly, synchronous cultures of P. falciparum Dd2-infected erythrocytes at 0.5% parasitemia and 2% hct were incubated with varying concentrations of Gleevec® and monitored every 11 hours (due to the Dd2 44 hour life cycle) for 143 hours corresponding to 4 life cycles. Untreated cultures were run in parallel as controls. Data are means of 2 samples per treatment condition.

Results

It was observed that during the first cycle (first 44 hours), Gleevec® did not cause significant changes in parasitemia of the culture. However, parasites treated with higher concentrations of Gleevec® fail to reinfect healthy erythrocytes at the second cycle. These results demonstrate that Gleevec® is effective as an antimalarial drug due to its off target effect as a Syk kinase inhibitor. These results are similar to the results seen when parasites are treated with other Syk kinase inhibitors. Parasites treated with higher concentrations of Gleevec® remain nonviable after analysis at what should be 4 cycles of growth.

Further, Gleevec, starting at 8 μM concentration, was able to delay the cycles of the parasite. The parasitemia of the 8 μM sample doesn't increase at the same time as the control, where you see an increase in parasitemia between 77 and 88 hpi. In contrast, the increase in parasitemia in the 8 μM sample was not evident until 88-99 hpi, when the parasites eventually egress and infect new RBCs.

### Example 5

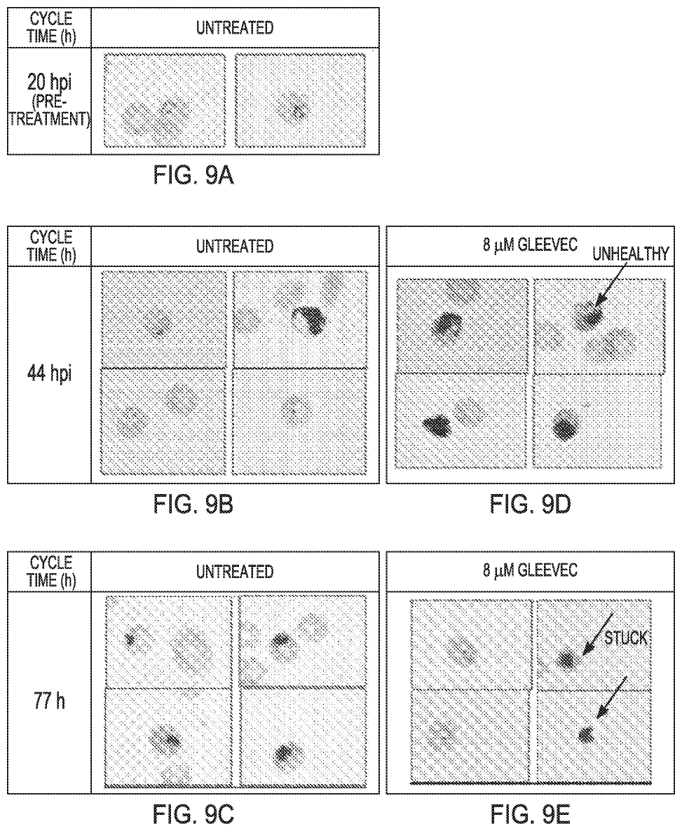

FIGS. 9A-9E are microscopy photographs of standard blood smears. Smears were prepared on glass slides, fixed with 100% methanol, and stained in 10% Giemsa modified stain diluted in PBS for 10 minutes at room temperature.

FIG. 9A are microscopy photographs of RBCs at 20 hours post infection (hpi). This time period corresponds to late rings, early trophs and is prior to treatment with Gleevec.

FIG. 9B are microscopy photographs of untreated RBCs at 44 hpi. At this time period, most schizonts had already egressed, and some schizonts remain. In contrast, RBCs treated with 8 μM Gleevec® at 44 hpi are still developing into early schizonts and mature schizonts (FIG. 9D). By comparing FIG. 9B and FIG. 9D, one can see that the delayed development of the parasite cycle has already started.

FIG. 9C are microscopy photographs of untreated RBCs at 77 hours, which corresponds to 33 hpi (next round of infection). Trophs in cycle 2 are seen.

FIG. 9E are microscopy photographs of RBCs treated with 8 μM Gleevec® at 77 hours. Half of the cell population are rings that formed. In addition, merozoites are stuck in schizonts from last egress. The cells appear shriveled as compared to untreated RBCs.

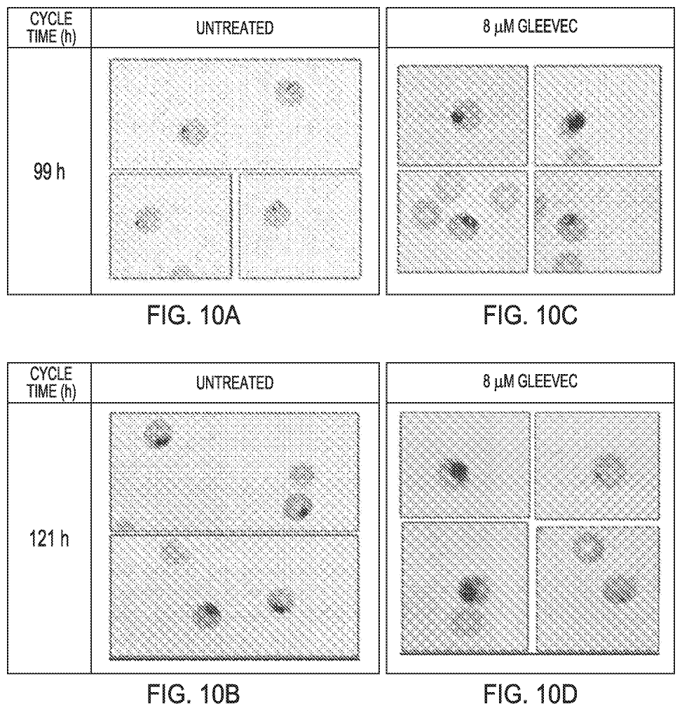

FIGS. 10A-D are microscopy photographs of standard blood smears. Smears were prepared on glass slides, fixed with 100% methanol, and stained in 10% Giemsa modified stain diluted in PBS for 10 minutes at room temperature.

FIG. 10A are microscopy photographs of untreated RBCs at 99 h, which corresponds to 11 bpi (next round). FIG. 10A shows that the rings have egressed for cycle 3 of the lifecycle. FIG. 10C are microscopy photographs of RBCs treated with 8 μM Gleevec® at 99 hours. FIG. 10C demonstrates that some cells have formed schizonts, but most are still stuck at trophs. Some of the RBCs look unhealthy; stipling of color can be seen in some RBCs around parasite as compared to other trophs in untreated RBCs, where they are nice round RBCs, with solid pigmentation.

FIG. 10B are microscopy photographs of untreated RBCs at 121 h, which corresponds to 33 bpi. Healthy trophs can be seen in FIG. 10B.

FIG. 10 D are microscopy photographs of RBCs treated with 8 μM Gleevec® at 121 hour. Schizonts from 2′ cycle are observed as well as some rings for cycle 3. Gleevec® is able to delay the lifecycle of the parasite.

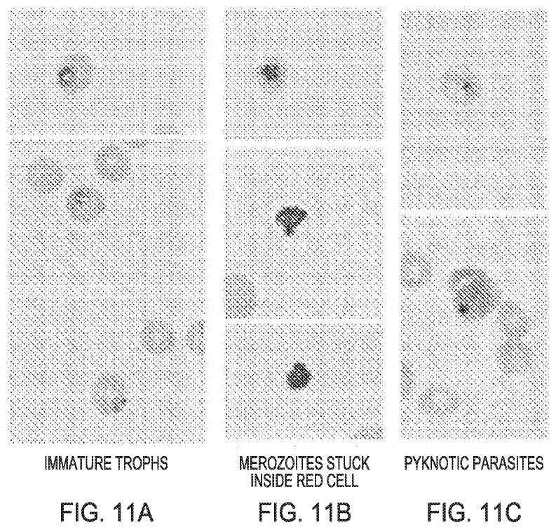

FIGS. 11A-C are microscopy photographs of standard blood smears. Smears were prepared on glass slides, fixed with 100% methanol, and stained in 10% Giemsa modified stain diluted in PBS for 10 minutes at room temperature.

FIG. 11A are microscopy photographs of RBCs treated with 10 μM Gleevec® and show immature trophs from the first cycle that have not matured.

FIG. 11B are microscopy photographs of RBCs treated with 10 μM Gleevec® and show merozoites stuck or trapped inside the RBCs that cannot egress.

FIG. 11C are microscopy photographs of RBCs treated with 10 μM Gleevec® and show pyknotic parasites, or dead parasites in condensed form.

Treatment of RBCs with Gleevec® blocks Syk-catalyzed band 3 tyrosine phosphorylation and prevents malaria induced membrane fragmentation. The use of Syk kinase inhibitors provides a strategy for treatment of malaria that the parasite cannot evade. The use of Syk kinase inhibitors also provides a therapeutic avenue for drug resistant malaria. The parasite does not have tyrosine kinases, and thus, the parasite cannot mutate the kinase to avoid the therapeutic intervention.

### Example 6

In this example, we analyzed the ability of an Syk kinase inhibitor to reduce parasitemia in blood from human subjects infected with the parasite. The Syk kinase inhibitor was not injected into the subjects. Blood was removed from the patient and then the inhibitor was added.

Materials and Methods

Blood collected from malaria infected Vietnamese patients was treated with varying concentrations of Syk kinase inhibitor II and analyzed through PCR. Upon arrival of each sample, malaria infected RBCs were separated from plasma and leukocytes by three washes in wash medium (RPMI 1640 medium containing 2 mM glutamine, 24 mM NaHCO, 25 mM Hepes, 20 mM glucose, and 32 mg/ml gentamicin, pH 6.80).

Smears of infected erythrocytes were obtained to assess initial parasitemia. Slides were fixed in 100% methanol and stained with 1/10 dilution of Giemsa solution, incubated for 10 min at room temperature, washed with water, dried, and observed under oil immersion lens (100×) for microscopic analysis. An aliquot was also saved for PCR analysis.

Malaria infected erythrocytes were re-suspended at 2% hematocrit in growth medium consisting of RPMI 1640 supplemented with 2 mM glutamine, 24 mM NaHCO, 25 mM Hepes, 20 mM glucose, and 32 mg/ml gentamicin, pH 6.80 and 10% heat inactivated human serum. Infected blood was aliquoted into 96 well plates pretreated with Syk kinase inhibitor H and incubated at 37° C. in CO₂ incubation bags. After 48 hours of incubation, the cultures were processed and analyzed by PCR using appropriate primers.

Results

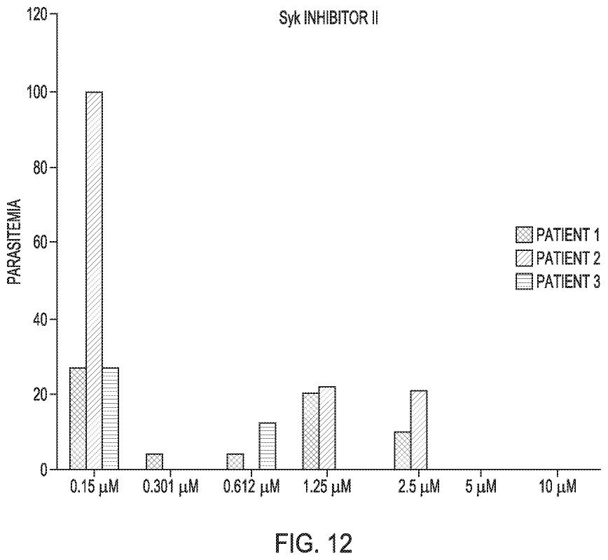

As shown in FIG. 12, Syk kinase inhibitor II was capable of completely eliminating parasitemia from infected blood acquired directly from Vietnamese patients. Various concentrations of Syk kinase inhibitor II were effective. In addition, Syk kinase inhibitor was effective in reducing parasitemia in numerous samples.

Syk kinase inhibitor II is effective as a novel, potent antimalarial drug.

### Example 7

In this example, we analyzed the ability of Gleevec® to reduce parasitemia in blood from human subjects infected with the parasite. Gleevec® was not injected into the subjects. Blood was removed from the subject and then the inhibitor was added.

Materials and Methods

Blood collected from malaria infected Vietnamese patients was treated with varying concentrations of Gleevec® and analyzed through PCR. Upon arrival of each sample, malaria infected RBCs were separated from plasma and leukocytes by three washes in wash medium (RPMI 1640 medium containing 2 mM glutamine, 24 mM NaHCO, 25 mM Hepes, 20 mM glucose, and 32 mg/ml gentamicin, pH 6.80).

Smears of infected erythrocytes were obtained to assess initial parasitemia. Slides were fixed in 100% methanol and stained with 1/10 dilution of Giemsa solution, incubated for 10 min at room temperature, washed with water, dried, and observed under oil immersion lens (100×) for microscopic analysis. An aliquot was also saved for PCR analysis.

Malaria infected erythrocytes were re-suspended at 2% hematocrit in growth medium consisting of RPMI 1640 supplemented with 2 mM glutamine, 24 mM NaHCO, 25 mM Hepes, 20 mM glucose, and 32 mg/ml gentamicin, pH 6.80 and 10% heat inactivated human serum. Infected blood was aliquoted into 96 well plates pretreated with Gleevec® and incubated at 37° C. in CO₂ incubation bags. After 48 hours of incubation, the cultures were processed and analyzed by PCR.

Results

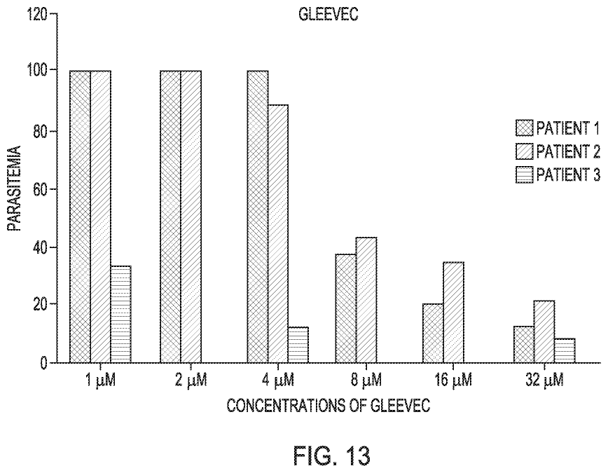

As shown in FIG. 13, Gleevec® is also effective at reducing the parasitemia of malaria infected blood taken directly from Vietnamese patients. Gleevec® functions as an Syk kinase inhibitor. Gleevec® is FDA approved and concentrations of 8 μM and 10 μM can be obtained in patients. In this experiment, blood was treated with one dose of Gleevec. Multiple doses would be effective at eradicating parasitemia.

### Example 8

Syk kinase inhibitors are useful as therapeutic agents for the treatment of drug resistant malaria. The enzyme that disrupts the interaction between band 3 and ankyrin, which are two proteins involved in cell membrane stabilization, belongs to the red blood cell. Thus, the parasite cannot mutate a kinase that is encoded by the host genome.

In addition, the parasite cannot mutate one of its own tyrosine kinases to phosphorylate band 3 because there are no tyrosine kinases in the entire parasite genome. Thus, Syk kinase inhibitors provide an effective therapeutic choice for which resistance is highly unlikely.

Materials and Methods

A blood sample was acquired from patient 4 prior to treatment with artemisinin and 13 day after treatment with artemisinin. The patient had an initial parasitemia of 0.1%. After artemisinin treatment, the patient still had a slight parasitemia. These parasites are considered artemisinin resistant since there is a persistent parasitemia in the patient, even after artemisinin therapy.

Upon arrival of each sample, malaria infected RBCs were separated from plasma and leukocytes by three washes in wash medium (RPMI 1640 medium containing 2 mM glutamine, 24 mM NaHCO, 25 mM Hepes, 20 mM glucose, and 32 mg/ml gentamicin, pH 6.80).

Smears of infected erythrocytes were obtained to assess initial parasitemia. Slides were fixed in 100% methanol and stained with 1/10 dilution of Giemsa solution, incubated for 10 min at room temperature, washed with water, dried, and observed under oil immersion lens (100×) for microscopic analysis. An aliquot was also saved for PCR analysis.

Malaria infected erythrocytes were re-suspended at 2% hematocrit in growth medium consisting of RPMI 1640 supplemented with 2 mM glutamine, 24 mM NaHCO, 25 mM Hepes, 20 mM glucose, and 32 mg/ml gentamicin, pH 6.80 and 10% heat inactivated human serum. Infected blood was aliquoted into 96 well plates pretreated with Syk kinase inhibitor II and incubated at 37° C. in CO₂ incubation bags. After 48 hours of incubation, the cultures were processed and analyzed by PCR.

Results

Syk kinase inhibitor II was able to completely eliminate parasites in malaria infected blood pre- and post-artemisinin treatment.

TABLE III
Syk kinase inhibitor II was able to eliminate parasitemia
in artemisinin resistant blood acquired directly
from an infected Vietnamese patient.

| | | Syk kinase |
|---|---|---|
| | Parasitemia | inhibitor II |
| Sample pre-treatment | 0.1% | No growth detectable |
| | | above 2.5 μM |
| Sample post-treatment | <0.001% | No growth detectable |
| (13 days) | | above 2.5 μM |

TABLE IV
IC₅₀ 0rSvk kinase inhibitor II.

| | | IC₅₀ Syk kinase |
|---|---|---|
| | Parasitemia | inhibitor II |
| Sample pre-treatment | 0.1% | <0.6 μM |

Since artemisinin resistance is spreading to areas of Vietnam, this is clinically relevant as there are no antimalarial therapies that can treat patients infected with these resistant malaria parasites. These results prove Syk kinase inhibitors are able to effectively eliminate artemisinin resistant parasites obtained directly from infected Vietnamese patients. Since the malaria parasite cannot modify endogenous host Syk kinase nor has tyrosine kinases of its own, malaria resistance to this therapy is not possible.

### Example 9

A Syk kinase inhibitor will be administered to a patient, wherein the patient has one or more of the following characteristics: (1) the patient has malaria or a condition similar to malaria; (2) the patient is suspected of being a carrier of malaria or a condition similar to malaria; (3) the patient has a drug resistant form of malaria or a condition similar to malaria; or (4) the patient is recovering from a recent episode of malaria.

In one embodiment, the Syk kinase inhibitor that will be administered is Gleevec. In another embodiment, Gleevec® will be administered from about 400 mg to about 1000 mg. In another embodiment, Gleevec® will be administered with one or more anti-malaria drugs.

In still another embodiment, Gleevec® will be administered with one or more additional Syk kinase inhibitors.

In still another embodiment, Gleevec® will be administered with one or more additional Syk kinase inhibitors and one or more anti-malaria drugs.

Although specific embodiments have been illustrated and described herein, it will be appreciated by those of ordinary skill in the art that any arrangement that is calculated to achieve the same purpose may be substituted for the specific embodiments shown. This application is intended to cover any adaptations or variations that operate according to the principles of the disclosure as described. Therefore, it is intended that this disclosure be limited only by the claims and the equivalents thereof. The disclosures of patents, references and publications cited in the application are incorporated by reference herein.

1-20. (canceled)

21. A method comprising administering at least one compound that inhibits band 3 phosphorylation in red blood cells to a subject in need thereof, wherein the at least one compound is not imatinib.

22. The method of claim 21, wherein the at least one compound inhibits band 3 tyrosine phosphorylation in red blood cells.

23. The method of claim 21, wherein the at least one compound inhibits band 3 tyrosine 21 phosphorylation in red blood cells.

24. The method of claim 21, wherein the subject is suffering from malaria.

25. The method of claim 24, wherein the malaria is drug-resistant malaria.

26. The method of claim 24, wherein malaria is Quartan malaria, Falciparum malaria, Biduoterian fever, Blackwater fever, Tertian malaria, Plasmodium, uncomplicated malaria and severe malaria.

27. The method of claim 21, wherein the at least one compound is at least one Syk kinase inhibitor.

28. The method of claim 27, wherein the Syk kinase inhibitor is at least one of a Syk kinase inhibitor 11 and a Syk kinase inhibitor IV.

29. The method of claim 21, wherein the compound is at least one of a purine-2-benzamine derivative, a pyrimidine-5-carboxamide derivative, a 1,6-naphthyridine derivative, BAY 61-3606, piceatannol, 3,4-dimethyl-10-(3-aminopropyl)-9-acridone oxalate, R406, and R788.

30. The method of claim 21, wherein the subject is a carrier of malaria.

31. The method of claim 21 further comprising administering an antimalarial drug in combination with the at least one compound to the subject.

32. The method of claim 31, wherein the antimalarial drug is a 4-aminoquinolone compound.

33. The method of claim 31, wherein the antimalarial drug is selected from the group consisting of artemisinin, chloroquine, quinine, mefloquine, and indolone N-oxides (INODS) of various structures.

34. The method of claim 21, wherein the at least one compound that inhibits band 3 phosphorylation in red blood cells inhibits malaria parasite egress from malaria parasite-infected red blood cells in a subject.

35. The method of claim 21, wherein the at least one compound that inhibits band 3 phosphorylation in red blood cells stabilizes red blood cells' membranes and reduces the incidence of malaria parasite egress from malaria-infected red blood cells in the subject.
